(19) United States
(12) Patent Application Publication
(10) Pub. No.: US 2026/0334608 A1
(43) Pub. Date: Oct. 29, 2026

(54) RESPIRATOR

(71) Applicant: Avon Polymer Products Limited, Melksham (GB)

(72) Inventors: Sebastian Parker, Melksham (GB); Nicholas Hunter, Melksham (GB)

(73) Assignee: Avon Polymer Products Limited, Melksham (GB)

(21) Appl. No.: 19/118,286

(22) PCT Filed: Oct. 3, 2023
(86) PCT No.: PCT/EP2023/077323

§ 371 Date: Apr. 3, 2025

(30) Foreign Application Priority Data
Oct. 3, 2022 (GB) 2214518.9

(51) Int. Cl. A41D 13/11; A61F 9/02; A62B 7/12; A62B 18/08; A62B 23/02

(52) U.S. Cl. CPC A41D 13/1184; A61F 9/029; A62B 18/082; A62B 23/02; A62B 7/12

(57) ABSTRACT

A mask for a respirator includes a nose cup for covering the wearer's nose and mouth, and a sealing arrangement configured to provide a seal between the mask and the face of the wearer. The sealing arrangement comprises a chin portion which is configured to extend over a wearer's chin and has an outer surface which is configured to follow the shape of the wearer's chin.

### CROSS-REFERENCE TO RELATED APPLICATIONS

This application is the U.S. national stage entry of PCT/EP2023/077323, filed on Oct. 3, 2023, which claims priority to United Kingdom Application No. 2214518.9, filed Oct. 3, 2022, the entire disclosures of which are fully incorporated herein by reference.

### TECHNICAL FIELD

The present disclosure relates to a respirator and particularly, although not exclusively, to a modular respirator. The present disclosure also relates to goggles and a mask, and in particular, although not exclusively, to goggles and a mask for use in a modular respirator. The present disclosure also relates to components for use in goggles, masks and/or respirators.

### BACKGROUND

A respirator mask is a device that is used to protect a wearer from dangerous materials or substances, for example chemical, biological, nuclear and radiological agents, and/or toxic industrial chemicals, and/or toxic industrial materials.

A respirator mask typically includes a nose cup that covers at least the nose and mouth of the user and provides a seal around the nose and mouth. The nose cup is provided either with a filter for filtering air inhaled into the nose cup by the user, or with a connector for connecting the nose cup to a supply of filtered air. The nose cup is also typically provided with an exhale valve through which exhaled air can be discharged from the nose cup.

A respirator mask also typically includes a visor or goggles portion that covers the eyes of the user and protects the eyes of the user. The visor or goggles portion typically provides a seal around the eyes of the user.

The present inventors have identified various problems with conventional respirator masks, including problems related to ease of use of conventional respirator masks.

The present disclosure has been devised in light of the above considerations.

### SUMMARY

According to a first aspect of the present disclosure there is provided protective goggles comprising a sealing arrangement configured to provide a substantially airtight seal between the protective goggles and a face of a wearer, and a pump arranged to pump air into an ocular space defined by the protective goggles.

According to the first aspect of the present disclosure, a positive pressure may be maintained in the ocular space, which may assist in keeping the ocular space free of contaminants.

The first aspect of the present disclosure may have any one, or, where compatible, any combination of the following optional features.

The sealing arrangement may provide an airtight seal between the protective goggles and the face of the wearer.

The sealing arrangement may alternatively be referred to as a seal, or one or more seals, or a sealing member, or one or more sealing members.

The ocular space may alternatively be referred to as an eye space.

The pump may comprise a miniature pump, or a micropump, or a piezoelectric pump, or a piezoelectric miniature pump, or a piezoelectric micropump.

A miniature pump or micropump may be particularly advantageous as it may require very little energy to operate.

A piezoelectric pump may be particularly advantageous as it may operate at a frequency close to, or above, an upper limit of human hearing, which may make the pump substantially silent, or silent.

The pump may comprise a fan.

The protective goggles may further comprise a visor or one or more eyepieces for protecting the wearer's eyes, wherein the ocular space is defined at least in part between the visor or one or more eyepieces and the sealing arrangement.

The protective goggles may further comprise a frame; wherein the visor or one or more eyepieces are connected to the frame; and wherein the ocular space is defined at least in part between the frame, the visor or one or more eyepieces, and the sealing arrangement.

The pump may be a piezoelectric micropump having an operating frequency of greater than 20 kHz.

The protective goggles may further comprise a pressure sensor for sensing an air pressure in the ocular space, and a controller configured to control the pump responsive to the sensed air pressure. The controller may be implemented via any suitable computing (or processing) device, such as a microcontroller. As an example, the controller may be configured to control the pump as a function of the sensed air pressure, to achieve (or maintain) a target (predetermined) air pressure in the ocular space. In this manner, the air pressure in the ocular space can be automatically controlled, using the sensed pressure from the pressure sensor as feedback. This may ensure that an adequate pressure is maintained in the ocular space to prevent contaminants in the environment from entering the ocular space, as well as maintain a comfortable pressure level for the user.

The protective goggles may further comprise a second pressure sensor for sensing an ambient air pressure, wherein the controller is configured to control the pump in response to a difference between the ocular space air pressure and the ambient air pressure. This may ensure that a suitable pressure differential between the ocular space and the ambient air is maintained. The first pressure sensor may be arranged (positioned) so that it is exposed to the ocular space in the protective goggles, whilst the second pressure sensor may be arranged (positioned) exposed so that it is exposed to ambient air outside the ocular space.

The protective goggles may further comprise a differential pressure sensor for measuring a difference between an ambient air pressure and an air pressure in the ocular space, and a controller configured to control the pump responsive to the measured difference in air pressure. Similarly to the above, this may ensure that a suitable pressure differential between the ocular space and the ambient air is maintained.

The pump may be fluidly connected to an air channel through which air is pumped into the ocular space, the air channel comprising an aperture configured to direct the air across an inside surface of a visor or one or more eyepieces of the protective goggles.

The air channel may be a channel within a frame of the protective goggles.

The protective goggles may further comprise an outlet valve for allowing air out of the ocular space.

The outlet valve may be configured to maintain the pressure in the ocular space at or above a predetermined pressure.

The outlet valve may be configured to maintain a pressure differential between the ocular space and a surrounding atmosphere that is equal to or more than a predetermined pressure differential.

The protective goggles may further comprise a filter arranged to filter air pumped into the ocular space by the pump.

The filter may be upstream of the pump.

The filter may comprise a filter media or filter material.

The protective goggles may further comprise a strap or mounting arrangement for retaining the protective goggles in place.

The strap or mounting arrangement may be secured to a head of the wearer, and/or to a helmet worn by the wearer.

The protective goggles may be connectable to a nose cup of a mask, and the pump may be configured to pump air from within the nose cup of the mask into the ocular space of the protective goggles.

The air in the nose cup may be filtered air. In particular, the mask may comprise a filter arranged to filter air breathed into the mask, or the mask may be connected to a source of filtered air.

According to a second aspect of the present disclosure there is provided a device comprising: a visor or eyepiece; and a defogging arrangement configured to generate and direct an airflow across an inside surface of the visor or eyepiece.

According to the second aspect of the present disclosure, fogging of the visor or eyepiece may be effectively prevented or reduced by directing airflow across the inside surface of the visor or eyepiece.

The second aspect of the present disclosure may have any one, or, where compatible, any combination of the following optional features.

The device of the second aspect may correspond to the protective goggles of the first aspect.

Accordingly, any feature described in relation to the first aspect of the disclosure may be shared with the second aspect of the disclosure (and vice versa).

The visor or eyepiece may be for protecting a wearer's eyes.

Directing the airflow across the inside surface of the visor or eyepiece may comprise directing the air flow at least partly parallel to a surface of the visor or eyepiece.

The device may be configured to be mounted on a face or a head of a user.

The visor or eyepiece may be optically clear.

The device may be for protection, i.e. protection of the eyes of the wearer, and/or vision enhancement, and/or displaying information.

The defogging arrangement may comprise an air channel through which the airflow passes, and an aperture in a wall of the air channel may be arranged to direct the airflow across the inside surface of the visor or eyepiece.

The aperture may comprise a nozzle for directing and/or accelerating the airflow.

The defogging arrangement may comprise a plurality of the apertures in the wall of the air channel spaced apart along the air channel.

The device may further comprise a frame, wherein the visor or eyepiece is connected to the frame and the air channel is a channel within the frame. The aperture or apertures may be apertures in a wall of the frame.

The defogging arrangement may comprise a fan, or a pump, or a piezoelectric pump, or a piezoelectric micropump, or a piezoelectric miniature pump, for generating the airflow.

The device may further comprise a sealing arrangement, configured to provide a substantially airtight seal, or an airtight seal, between the device and the face or head of a wearer.

The device may further comprise a filter arranged to filter the airflow.

The device may further comprise a strap or mounting arrangement for mounting the device on the face or head of a wearer.

The device may be protective goggles. Where the device is the protective goggles of the first aspect of the disclosure, the defogging arrangement may include (or correspond to) the pump of the protective goggles.

According to a third aspect of the present disclosure there is provided a respirator comprising: protective goggles for protecting a wearer's eyes; and a mask for covering a wearer's nose and mouth; wherein the protective goggles and the mask are detachably connectable to one another.

According to the third aspect of the present disclosure, the respirator is a modular respirator in which the protective goggles and mask can be used together or independently and wherein they can be connected together and detached from one another.

The third aspect of the present disclosure may have any one, or, where compatible, any combination of the following optional features.

The protective goggles may, for example, correspond to the protective goggles of the first aspect of the disclosure described above. Accordingly, any feature described in relation to the first aspect of the disclosure may be shared with the third aspect of the disclosure (and vice versa). Similarly, features of the second aspect of the disclosure can be shared with the third aspect of the disclosure (and vice versa).

The protective goggles and the mask may be detachable from one another while in use, for example while being worn.

The protective goggles may comprise a first connector and the mask may comprise a second connector, wherein the first connector and the second connector are detachably connectable together to detachably connect the protective goggles and the mask to one another.

The first connector and the second connector may be configured to allow relative movement between the protective goggles and the mask when the first and second connectors are connected together. This can enable relative positions of the protective goggles and the mask to be adjusted by the user for improved comfort. Additionally, allowing relative movement between the protective goggles and the mask enables the respirator to be adapted to different shapes and sizes of face. As an example, the first connector and the second connector may be configured to allow the protective goggles and the mask to pivot relative to one another when the first and second connectors are connected together. Additionally or alternatively, the first connector and the second connector may be configured to allow a distance between the protective goggles and the mask to be varied when the first and second connectors are connected together. Various types of connectors can be used to provide this functionality, examples of which are provided below.

A first one of the first connector and the second connector may comprise a hook.

A second one of the first connector and the second connector may comprise a catch or loop or ring or aperture or opening for connecting to the hook.

In this manner, the hook can be engaged in the catch (or loop or ring or aperture) to connect the protective goggles and the mask together. Whilst the hook is engaged in the catch (or loop or ring or aperture), the hook may be moveable (e.g. pivotable) within the catch, to allow a certain amount of relative movement between the protective goggles and the mask.

The second one of the first connector and the second connector may be a loop which extends outwardly away from the protective goggles or mask. In other words, where the loop is on the protective goggles, the loop may extend outwardly away from the protective goggles; where the loop is on the mask, the loop may extend outwardly away from the mask. This can facilitate engaging the hook in the loop. Additionally, with the loop extending (protruding) in the outward directing, this enables the protective goggles and the mask to be pivoted relative to one another when the hook is engaged in the loop, e.g. so that an angle between the mask and the protective goggles can be adjusted.

The hook may extend in a first direction away from the protective goggles or mask, and an open portion of the hook may face in a second direction at least partly opposite to the first direction. In other words, where the hook is on the protective goggles, the hook may extend in the first direction away from the protective goggles and the open portion of the hook may face in the second, opposite direction. Where the hook is on the mask, the hook may extend in the first direction away from the mask and the open portion of the hook may face in the second, opposite direction. Such an arrangement of the hook facilitates connecting the hook to the loop due to the hook extending in the first direction. However, as the open portion of the hook is in the second, opposite direction, this avoids the hook from being pulled out of the loop along the first direction. For example, in order to remove the hook from the loop, the hook may need to be pivoted relative to the loop.

As an example, the mask may comprise the hook and the hook may extend upwards away from the protective goggles towards the mask when the mask is worn by the user. The open portion of the hook may therefore face in a second direction that is at least partly downwards, for example downwards or at an angle downwards.

The first connector and second connector may be provided at a nose bridging portion of the protective goggles and the mask, respectively.

As an example, the protective goggles may comprise the loop, and the loop may be provided in the nose bridging portion of the protective goggles. Thus, the loop may extend around part of the wearer's nose when the protective goggles are worn. In this manner, the loop may be easily accessible for connecting the mask to the protective goggles.

The protective goggles and mask may further comprise a magnetic connector configured to provide an additional detachable connection between the protective goggles and mask. For example, a first magnetic connector may be provided in the protective goggles, and a second magnetic connector may be provided in the mask, wherein the first and second magnetic connectors are arranged to attract one another when the protective goggles and the mask are connected to one another. This may serve to strengthen the connection between the protective goggles and the mask. In some cases the first magnetic connector may be provided in the first connector, and the second magnetic connector may be provided in the second connector. The first and second magnetic connectors may comprise first and second magnets respectively.

According to a fourth aspect of the present disclosure there is provided a respirator comprising: protective goggles for protecting a wearer's eyes, comprising a goggle sealing arrangement configured to provide a seal between the protective goggles and the face of a wearer; and a mask for covering a wearer's nose and mouth, comprising a mask sealing arrangement configured to provide a seal between the mask and the face of a wearer, wherein the goggle sealing arrangement and the mask sealing arrangement comprise mutually overlapping portions.

According to the fourth aspect of the present disclosure, a more effective and/or comfortable seal between the respirator and the face of the user may be provided in the mutually overlapping portions.

The respirator according to the fourth aspect of the present disclosure may have any one, or, where compatible, any combination of the following optional features.

The protective goggles may, for example, correspond to the protective goggles of the first aspect of the disclosure described above. Accordingly, any feature described in relation to the first aspect of the disclosure may be shared with the fourth aspect of the disclosure (and vice versa). Similarly, features of the second aspect of the disclosure can be shared with the fourth aspect of the disclosure (and vice versa).

The protective goggles and the mask may be detachably connected to one another, as described above in relation to the third aspect of the disclosure. Accordingly, any feature described in relation to the third aspect of the disclosure can be shared with the fourth aspect of the disclosure (and vice versa).

The mutually overlapping portions may mutually overlap when both the protective goggles and mask are worn by the wearer. Thus, where the protective goggles and the mask are detachably connectable to one another, when the protective goggles and the mask are connected, the goggle sealing arrangement and the mask sealing arrangement comprise portions that mutually overlap one another. This serves to improve a quality of the seal between the protective goggles and the mask, to avoid any leaks at the interface between the protective goggles and the mask. Accordingly, the mutually overlapping portions may serve to form a substantially uniform (or continuous) seal with the wearer's face.

The mutually overlapping portions may be configured to mutually overlap when both the protective goggles and mask are worn by the wearer.

Mutually overlapping means that a portion of one of the goggle sealing arrangement and the mask sealing arrangement overlaps a portion of the other one of the goggle sealing arrangement and the mask sealing arrangement.

The mask sealing arrangement and the goggle sealing arrangement may each comprise a seal, or one or more seals, or a sealing member, or one or more sealing members. For example, the goggle sealing arrangement may comprise a seal (sealing member) that extends around an edge of the protective goggles, and which is arranged to form a seal against the user's face when in use, e.g. around the user's eyes. The mask sealing arrangement may comprise a seal (sealing member) that extends around an edge of the mask, and which is arranged to form a seal against the user's face when in use, e.g. around the user's nose and mouth.

In the mutually overlapping portions, the goggle sealing arrangement and the mask sealing arrangement may have complementary, tessellating, or congruous shapes and/or tangential surfaces. In other words, the mask sealing arrangement and the goggle sealing arrangement may have corresponding (e.g. complementary) geometries in the mutually overlapping portions.

A portion of the goggle sealing arrangement may have a shape that is complementary to a portion of the mask sealing arrangement, to provide a tight fit between the goggle sealing arrangement and the mask sealing arrangement, e.g. when the protective goggles and the mask are connected together.

In the mutually overlapping portions, a sealing surface of the goggle sealing arrangement may be substantially continuous (or continuous) with a sealing surface of the mask sealing arrangement, and/or tangential to a sealing surface of the mask sealing arrangement. In this manner, a continuous sealing line can be formed between the goggle sealing arrangement and the mask sealing arrangement, e.g. when the protective goggles and the mask are connected together.

In the mutually overlapping portions, a sealing surface of the goggle sealing arrangement and a sealing surface of the mask sealing arrangement may be configured to form a continuous contact line or surface with the face of the wearer. The continuous contact line or surface may thus form a continuous seal between the protective goggles and the mask, and with the wearer's face. In other words, a seal may be formed in the mutually overlapping portions between the goggle sealing arrangement and the mask sealing arrangement, and between the mutually overlapping portions and the wearer's face.

The continuous contact line or surface may be arranged to extend along an interface between the protective goggles and the mask. This may ensure that a seal is formed at the interface between the two parts.

The mutually overlapping portions may be located at least at nasal bridge regions of the protective goggles and the mask. This may facilitate forming a seal at the interface between the protective goggles and the mask. For example, the continuous contact line or surface mentioned above may be arranged to continuously extend around (or over) a bridge of a wearer's nose, e.g. from a left side of the wearer's nose to a right side of the wearer's nose. In some cases, the continuous contact line or surface may be arranged to extend along part of the wearer's cheeks (e.g. under the wearer's eyes). Thus, when the protective goggles and the mask are connected together, the mutually overlapping portions of the goggle sealing arrangement and the mask sealing arrangement can form a continuous sealing line or surface arranged to continuously extend around (or over) a bridge of the wearer's nose, and optionally along part of the wearer's cheeks.

The mutually overlapping portions may provide a layered structure.

The mask sealing arrangement and/or the goggles sealing arrangement may comprise one or more of: a resiliently deformable material; and an elastomeric material.

In the mutually overlapping portions, the mask sealing arrangement and the goggle sealing arrangement may form a substantially airtight seal, or airtight seal, therebetween.

According to a fifth aspect of the present disclosure there is provided a mask comprising: a nose cup for covering the wearer's nose and mouth; and a sealing arrangement configured to provide a seal between the mask and the face of the wearer, wherein the sealing arrangement comprises a chin portion which is configured to extend over a wearer's chin and has an outer surface which is configured to follow the shape of the wearer's chin.

According to the fifth aspect of the present disclosure, an external shape of the chin portion may correspond to an external shape of the wearer's chin. Therefore, the mask may be used with a helmet having a conventional helmet chin strap, for example.

The fifth aspect of the present disclosure may have any one, or, where compatible, any combination of the following optional features.

The mask may be for protecting the wearer from inhaling harmful substances.

The mask of the fifth aspect of the disclosure may be used as part of the respirator of the third and/or fourth aspect of the disclosure. Accordingly, any feature described in relation to the fifth aspect of the disclosure can be shared with the third and fourth aspect of the disclosure (and vice versa). The nose cup may be configured to extend from the top of the wearer's nose to the top of the wearer's chin, and the chin portion may be configured to extend from the top of the wearer's chin to underneath the wearer's chin. In other words, the chin portion may include a font portion arranged to be located on a front of the wearer's chin, and a lower portion arranged to be located under the wearer's lower jaw. Providing a sealing arrangement with a chin portion that extends from the top of the wearer's chin to underneath the wearer's chin may contribute to improving a sealing performance of the sealing arrangement, by increasing a contact area with the wearer's skin.

The chin portion may be configured to conform to the shape of the wearer's chin. For example, the chin portion may have a curved shape, so as to conform to the shape of the wearer's chin. Additionally, the chin portion may be formed of a resiliently deformable material, e.g. an elastomeric material, to facilitate conforming the chin portion to the shape of the wearer's chin.

A sealing surface of the chin portion may be tangential to, or continuous with, a sealing surface of another part of the sealing arrangement.

The sealing arrangement may comprise a protruding portion which protrudes from the chin portion to the nose cup. The protruding portion thus serves to join the chin portion to the nose cup.

The protruding portion may partially overlap the chin portion to form a recess or folded portion or pleat. This arrangement of the protruding portion may enhance a flexibility of the protruding portion, thus facilitating relative movement between the chin portion and the nose cup. This may be particularly beneficial where the protective goggles and the mask are detachably connected, as this may allow some relative movement between the protective goggles and the mask. For instance, the protruding portion can flex or bend to accommodate different positions of the nose cup (and hence the mask) on the wearer's face.

The protruding portion may be resiliently deformable and/or resiliently compressible.

The recess or folded portion or pleat may be resiliently deformable and/or resiliently compressible.

The protruding portion may comprise a bellows portion.

The recess or folded portion or pleat may comprise or form a bellows portion.

The recess or folded portion or pleat may be for receiving a chin strap of a helmet. Accordingly, in addition to providing enhanced flexibility of the chin portion, the recess or folded portion or pleat provides a convenient location for receiving the chin strap of a helmet. This can avoid the chin strap interfering with comfortable wearing of the mask and permits the wearer to don (fit) the mask without removing the helmet. Additionally, this may ensure that the chin strap does not slip out of place, such that the helmet can be reliably held on the wearer's head.

The nose cup may be a quarter mask nose cup.

The mask may further comprise a tab connected to the chin portion for gripping or holding by a user. For example, the tab may facilitate the user pulling the chin portion of the mask over their chin and/or removing the mask.

The mask may further comprise a filter for filtering air breathed into the mask.

The filter may comprise a one-way valve for allowing air flow from the filter into the mask but preventing airflow in an opposite direction.

According to a sixth aspect of the present disclosure there is provided a connector for detachably mounting a filter on a mask, the connector comprising: a main body having an aperture through which air can flow; a resiliently deformable clip mechanism comprising jaws that are configured to engage a mounting portion, wherein the clip mechanism is resiliently deformable so that the jaws can be moved out of engagement with the mounting portion, and wherein the clip mechanism comprises an actuator that is movable relative to the main body to deform the clip mechanism to move the jaws.

According to the sixth aspect of the present disclosure, the connector may be connected or mounted on the mounting portion by pushing the connector towards the mounting portion. The connector may therefore be a push-to-fit or push-fit connector. This may facilitate mounting of a filter on a mask. To remove the connector from the mounting portion it may be necessary to move the actuator relative to the main body to deform the clip mechanism, for example by pushing the actuator. Therefore, it may not be possible merely to pull the connector from the mounting portion, which may increase safety. However, it may still be relatively easy to remove the connector from the mounting portion.

The sixth aspect of the present disclosure may have any one, or, where compatible, any combination of the following optional features.

The actuator may be movable relative to the main body, for example by pushing the actuator and/or by squeezing the clip mechanism.

The jaws may be movable relative to the main body.

The jaws may be configured to engage the mounting portion therebetween, the clip mechanism may be resiliently deformable so that the jaws can be moved or splayed apart, and the actuator may be movable relative to the main body to deform the clip mechanism to move or splay the jaws apart.

Each of the jaws may comprise a projecting lip or rim, for example an inwardly projecting rim or lip.

Each of the jaws may have a chamfered lower surface.

The clip mechanism may comprise two jaws arranged to oppose each other.

Each of the jaws may be curved.

Each of the jaws may comprise a first end connected to the main body and a second end connected to the actuator.

The jaws and the actuator may be a unitary component and/or integral.

The clip mechanism may comprise a length of resiliently deformable material which is connected at each of its ends to the main body and which passes around the main body.

The length of resiliently deformable material may be connected at each of its ends to opposite ends of an arcuate section of the main body at an outer edge of the main body.

The clip mechanism may be squeezable so as to move the actuator relative to the main body.

The clip mechanism may be squeezable in a direction substantially perpendicular (or perpendicular) to a direction along which the jaws are configured to engage the mounting portion.

The main body may be a rigid body.

The connector may comprise a magnet.

There may also be provided a filter having the connector, wherein the jaws are configured to engage a mounting portion of a mask.

There may also be provided a mask having the connector, wherein the jaws are configured to engage a mounting portion of a filter.

The mounting portion may comprise an upstanding member having a projecting lip or rim, for example an outwardly projecting lip or rim.

The connector and mounting portion may both comprise magnets for magnetically connecting the connector to the mounting portion.

According to a seventh aspect of the present disclosure there is provided a filter for a mask, the filter comprising: a filter connector for connecting the filter to a filter mount of the mask; and a valve having a first configuration in which the valve does not block flow of air through the filter connector, and a second configuration in which the valve blocks flow of air through the filter connector; wherein the valve is changeable from the first configuration to the second configuration by a user pressing the valve.

According to the seventh aspect of the present disclosure, flow of air through the filter connector, and therefore flow of air through the filter into the mask through the filter mount of the mask, can be prevented by the user pressing the valve to change it into the second configuration. This may facilitate the user performing a negative pressure test of the mask, in which the user needs to prevent air being sucked into the mask through the filter and suck air from inside the mask to see if the mask is leaking in any places. In particular, the user can easily prevent air from being sucked into the mask through the filter by pressing the valve of the filter.

The seventh aspect of the present disclosure may have any one, or, where compatible, any combination of the following optional features.

The valve may be changeable from the first configuration to the second configuration by the user pressing the valve to displace part of the valve, for example to displace a sealing member of the valve.

The valve may be built into, or integral with, or connected to, or part of, the filter connector.

The valve may be biased towards the first configuration when the valve is in the second configuration.

The valve may comprise: a valve seat that is connected to, or integral with, the filter connecter so that air can only flow through the filter connector via the valve seat; and a sealing member configured to contact the valve seat in the second configuration to seal the valve.

The valve may further comprise one or more biasing elements for biasing the sealing member apart from the valve seat.

In the first configuration the sealing member may be spaced apart from the valve seat; and in the second configuration the sealing member may be pressed into contact with the valve seat.

The valve seat, the sealing member and the one or more biasing elements may be integrally formed, and/or one-piece, and/or unitary, and/or a single part.

The valve may comprise a plurality of the biasing elements that are spaced apart around circumferences of the valve seat and sealing member so that air can flow between the one or more biasing elements when the valve is in the first configuration.

The one or more biasing elements may comprise resilient hinges, or living hinges, or integral hinges.

The filter may further comprise filter media arranged upstream of the filter connecter in an air flow through the filter.

According to an eight aspect of the present disclosure there is provided a device comprising: a sealing arrangement configured to provide a seal between the device and the face of a wearer; wherein part of the sealing arrangement is inflatable to provide an improved seal between the device and the face of the wearer.

According to the eighth aspect of the present disclosure, the seal between the device and the face of the wearer can be improved by inflating the part of the sealing arrangement. Therefore, the wearer can be better protected by the device.

The eighth aspect of the present disclosure may have any one, or, where compatible, any combination of the following optional features.

The sealing arrangement may comprise a seal, or more or more seals, or a sealing member, or one or more sealing members.

The sealing arrangement may be configured to provide a seal between the device and the face of the wearer when the part of the seal is not inflated. Therefore, inflating the part of the seal may improve the existing seal.

The sealing arrangement may comprise an inflatable member that is inflatable to provide the improved seal between the device and the face of the wearer.

The inflatable member may comprise an inflatable bladder, bag, or bellows.

Part of the sealing arrangement that provides a seal between the device and the nose of the wearer may be inflatable.

The device may comprise a pump, for example a piezoelectric micropump or a fan, arranged to pump air into the part of the sealing arrangement.

The device may comprise a controller configured to control an operation of the pump.

The controller may be configured to control the pump to inflate the part of the sealing arrangement while the device is being worn by a wearer.

The controller may be configured to control the pump to pump air into the part of the sealing arrangement in response to an operation of the wearer, for example the user pressing an input device or button of the device.

The device may comprise a sensor for sensing a pressure in the part of the sealing arrangement, or a pressure in a sealed space of the device such as an ocular space, or a pressure difference between a sealed space of the device such as an ocular space and the surroundings of the device; and the controller may be configured to control an operation of the pump based on an output of the pressure sensor.

The part of the sealing arrangement may comprise a valve which is operable to deflate the part of the sealing arrangement.

The controller may be configured to control the valve, for example to open or close the valve, for example in response to an output of a pressure sensor as discussed above.

The device may be a head or face mountable device.

The device may be personal protective equipment.

The device may be protective goggles or a mask.

The device may be protective goggles, and the protective goggles may further comprise a frame and a visor or one or more eyepieces connected to the frame for protecting a wearer's eyes.

The device may comprise a strap or mounting arrangement for mounting the device on the head or face of the wearer.

The device may be a mask, and the mask may further comprise a filter for filtering air breathed into the mask.

The mask may further comprise an outlet valve, for example an exhale valve.

According to a ninth aspect of the present disclosure there is provided a device comprising: a sealing arrangement configured to provide a seal between the device and the face of a wearer; a strap or mounting arrangement for attaching the device to the head or face of the wearer, or for mounting the device on the head or face of the wearer; and an inflatable element that is arranged to apply outward pressure to the strap or mounting arrangement when inflated to provide an improved seal between the device and the face of the wearer.

The ninth aspect of the present disclosure may have any of the features of the eighth aspect of the present disclosure, unless incompatible.

The ninth aspect of the present disclosure may have any one, or, where compatible, any combination of the following optional features.

The inflatable element may be configured or arranged to be between a fixed part of the device and the strap or mounting arrangement, or between a head of the wearer and the strap or mounting arrangement.

The inflatable element may be part of, or attached to, or connected to, the strap or mounting arrangement.

The sealing arrangement may comprise a seal, or more or more seals, or a sealing member, or one or more sealing members.

The sealing arrangement may be configured to provide a seal between the device and the face of the wearer when the inflatable element is not inflated. Therefore, inflating the inflatable element may improve the existing seal.

The inflatable element may be an inflatable member or inflatable part.

The inflatable member may comprise an inflatable bladder, bag, or bellows.

The device may comprise a pump, for example a piezoelectric micropump, arranged to pump air into the inflatable element.

The device may comprise a controller configured to control an operation of the pump.

The controller may be configured to control the pump to inflate the inflatable element while the device is being worn by a wearer.

The controller may be configured to control the pump to pump air into the inflatable element in response to an operation of the wearer, for example the user pressing an input device or button of the device.

The device may comprise a sensor for sensing a pressure in the inflatable element, or a pressure in a sealed space of the device such as an ocular space, or a pressure difference between a sealed space of the device such as an ocular space and the surroundings of the device; and the controller may be configured to control an operation of the pump based on an output of the pressure sensor.

The inflatable element may comprise a valve which is operable to deflate the inflatable element.

The controller may be configured to control the valve, for example to open or close the valve, for example in response to an output of a pressure sensor as discussed above.

The device may be a head or face mountable device.

The device may be personal protective equipment.

The device may be protective goggles or a mask.

The device may be protective goggles, and the protective goggles may further comprise a frame and a visor or one or more eyepieces connected to the frame for protecting a wearer's eyes.

The device may comprise a strap or mounting arrangement for mounting the device on the head or face of the wearer.

The device may be a mask, and the mask may further comprise a filter for filtering air breathed into the mask.

The mask may further comprise an outlet valve, for example an exhale valve.

According to a tenth aspect of the present disclosure there is provided a flexible mounting arrangement or harness for a mask, comprising: a plenum comprising a flexible air permeable structure; and a connector for connecting the mounting arrangement or harness to a filter mount of the mask, wherein the connector is in fluid communication with the plenum; wherein the plenum is enclosed, or substantially enclosed, at least partly by filter material.

According to the tenth aspect of the present disclosure, a flexible mounting arrangement or harness for the mask that also functions as the filter for the mask is provided. This may facilitate mounting the mask on the face or head of a user and/or increase a surface area of filter material available to filter air breathed into the mask.

The tenth aspect of the present disclosure may have any one, or, where compatible, any combination of the following optional features.

Flexible may mean that the mounting arrangement can be bent or deformed without breaking or being permanently bent or deformed.

A plenum may mean a plenum space.

A plenum may mean an enclosed space or volume along which, or through which, air can flow.

The plenum may be defined by the flexible air permeable structure and the material or materials enclosing the flexible air permeable structure.

The plenum may therefore correspond to a volume enclosed by the material or materials that enclose the plenum, wherein the volume is at least partly occupied by the flexible air permeable structure.

An air permeable structure means a structure, typically a three-dimensional structure, through which or along which air can flow.

There may therefore be one or more air flow paths existing along or through the air permeable structure.

Fluid communication may mean gas communication, or that gas can flow from the connector from the plenum.

The plenum may be enclosed, or substantially enclosed, by filter material. For example, the plenum may be entirely or fully enclosed by filter material. Alternatively, the plenum may be enclosed, or substantially enclosed, by both filter material and one or more additional materials that is not a filter material. Such additional material may be a gas and/or a gas and liquid impermeable material.

The filter material may comprise filter media.

The filter material may be in fluid communication with the plenum.

The flexible mounting arrangement or harness may comprise a laminate structure comprising the air permeable structure sandwiched between an inner layer and an outer layer, and the inner layer and/or the outer layer may comprise filter material.

The inner layer and/or the outer layer may be at least partly made of filter material.

The air permeable structure may be resilient.

The air permeable structure may be resiliently deformable.

The air permeable structure may comprise one or more of: a 3D spacer material; a 3D spacer fabric; expanded paper; an open cell foam; a 3D printed lattice; and a chainmail.

The filter material may comprise one or more of: a fluoroplastic porous film; an electret; a spunbound or meltblown material; and expanded PTFE.

The mounting arrangement or harness may be configured to extend from the mask along both sides of a wearer's head to the back of the wearer's head.

The flexible mounting arrangement or harness may further comprise one or more fasteners for fastening the flexible mounting arrangement or harness around the wearer's head.

The flexible mounting arrangement or harness may further comprise a layer of material on an external surface of the filter material.

The layer of material may be hydrophobic and/or have a hydrophobic layer, coating or finish.

The plenum may not be enclosed where the connector is provided.

There may also be provided a mask comprising: a filter mount; and the mounting arrangement or harness connected to the filter mount.

The mask may further comprise a nose cup, and air may be breathable into the nose cup by the wearer via the filter mount.

The filter mount may be a mount for mounting a filter on the mask so that air can be breathed into the mask via the filter.

According to an eleventh aspect of the present disclosure there is provided a flexible mounting arrangement or harness for a mask, comprising: a plenum comprising a flexible air permeable structure; a first connector for connecting the mounting arrangement or harness to a filter mount of the mask, wherein the first connector is in fluid communication with the plenum; a second connector for connecting the mounting arrangement or harness to an air supply, wherein the second connector is in fluid communication with the plenum; and a gas impermeable material enclosing, or substantially enclosing, the plenum.

According to the eleventh aspect of the present disclosure, a flexible mounting arrangement or harness for the mask also functions as a means for connecting an air supply, typically a filtered air supply, to the mask.

The eleventh aspect of the present disclosure may have any of the features of the eighth aspect of the present disclosure described above, unless incompatible.

The air supply may comprise a tank or container of air, which may be pressurised.

The gas impermeable material may be a gas and liquid impermeable material.

The gas (or gas and liquid) impermeable material may comprise one or more of: an elastomeric material; rubber, for example thermoset or thermoplastic rubber; a sealed fabric; polyurethane; and Butyl rubber.

The second connector may be for connecting the flexible mounting arrangement or harness to a breathing hose.

The flexible mounting arrangement or harness may comprise a laminate structure comprising the air permeable structure sandwiched between an inner layer of gas impermeable material and an outer layer of gas impermeable material.

The air permeable structure may be resilient.

The air permeable structure may comprise one or more of: a 3D spacer material; a 3D spacer fabric; expanded paper; an open cell foam; a 3D printed lattice; and a chainmail.

The mounting arrangement or harness may be configured to extend from the mask along both sides of a wearer's head to the back of the wearer's head.

The flexible mounting arrangement or may further comprise one or more fasteners for fastening the flexible mounting arrangement or harness around the wearer's head.

The plenum may not be enclosed where the connector is provided.

There may also be provided a mask comprising: a filter mount; and the mounting arrangement or harness of any one of the preceding claims connected to the filter mount.

The disclosure includes the combination of the aspects and preferred features described except where such a combination is clearly impermissible or expressly avoided.

References herein to a mask may mean a protective mask, and/or breathing mask and/or a respirator mask, and/or filtration mask, for example.

References herein to a respirator may mean a respirator mask, or respirator device, for example.

### SUMMARY OF THE FIGURES

Embodiments and experiments illustrating the principles of the disclosure will now be discussed with reference to the accompanying figures in which:

FIG. 1 shows a perspective view of a user wearing a respirator according to an embodiment of the present disclosure;

FIG. 2 shows a perspective view of goggles for a respirator according to an embodiment of the present disclosure;

FIG. 3 is a schematic drawing of a defogging arrangement which may be used in embodiments of the present disclosure;

FIG. 4 is a perspective view of goggles for a respirator according to an embodiment of the present disclosure;

FIG. 5 is a cross-section view of a respirator according to an embodiment of the present disclosure;

FIG. 6 is a cross-section view of a mask for a respirator according to an embodiment of the present disclosure;

FIG. 7 is a rear view of a respirator according to an embodiment of the present disclosure;

FIG. 8 is a cross-section view of a respirator according to an embodiment of the present disclosure;

FIG. 9 is a perspective view of a user wearing a mask according to an embodiment of the present disclosure;

FIG. 10 shows a bottom view of a filter clip according to an embodiment of the present disclosure;

FIG. 11 shows a cross-section view of the filter clip connected to a filter mount;

FIG. 12 shows a perspective view of a filter clip according to an embodiment of the present disclosure;

FIG. 13 shows a perspective view of a negative pressure test valve according to an embodiment of the present disclosure;

FIG. 14 shows a cross-section view of the negative pressure test valve in an open configuration;

FIG. 15 shows a cross-section view of the negative pressure test valve in a closed configuration;

FIG. 16 shows a rear view of a harness according to an embodiment of the present disclosure;

FIG. 17 shows a schematic drawing of a harness according to an embodiment of the present disclosure;

FIG. 18 shows a perspective view of a user wearing a respirator according to an embodiment of the present disclosure that includes a harness;

FIG. 19 shows a schematic drawing of a harness according to an embodiment of the present disclosure;

FIG. 20 shows a cross-section view of a harness incorporating a filter clip and a negative pressure test valve wherein the filter clip is connected to a filter mount;

FIG. 21 shows the arrangement of FIG. 20 with the negative pressure test valve in a closed position;

FIG. 22 shows a cross-section view of a respirator incorporating an inflatable bladder;

FIG. 23 shows the arrangement of FIG. 22 with the inflatable bladder in a deflated condition;

FIG. 24 shows a perspective view of a respirator strap incorporating an inflatable bladder; and

FIG. 25 shows the arrangement of FIG. 24 with the inflatable bladder in a deflated condition.

### DETAILED DESCRIPTION

Aspects and embodiments of the present disclosure will now be discussed with reference to the accompanying figures. Further aspects and embodiments will be apparent to those skilled in the art. All documents mentioned in this text are incorporated herein by reference.

### Respirator

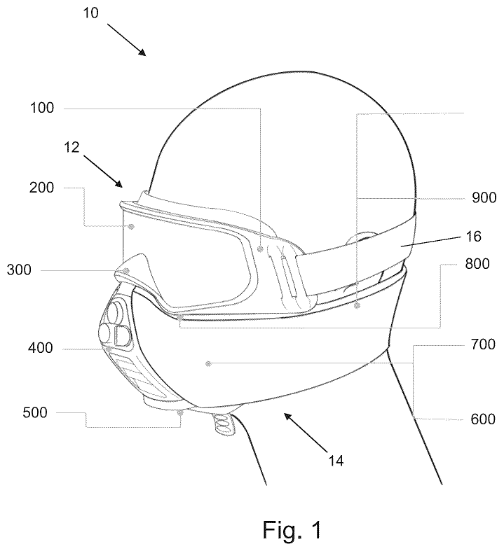

FIG. 1 shows a perspective view of a respirator 10 according to an embodiment of the present disclosure, showing the respirator 10 as worn by a user. For example, the respirator 10 may be used as part of chemical, biological, radiological and nuclear (CBRN) personal protective equipment. As explained in more detail below, the respirator 10 comprises a number of features which improve the usability and/or performance of the respirator 10.

The respirator 10 comprises goggles 12, which provide eye protection for a wearer, and a mask 14, which is configured to cover a wearer's nose and mouth to provide protection against gases, vapours and/or airborne particles when a wearer breathes.

The goggles 12 may be referred to as safety goggles or protective goggles, for example. The goggles 12 are configured to shield the eyes of a person wearing the goggles 12 against one or more of vapours, fumes and liquid droplets. In particular, the goggles 12 are configured to form a seal around the eyes of the person wearing the goggles 12. The goggles 12 comprise a seal configured to form the seal around the eyes of the person wearing the goggles 12. The goggles 12 are held in place over the eyes of the person using a strap 16 around the head of the person. The strap 16 may be referred to as a head strap, for example. In other examples, the goggles 12 may be connected directly or indirectly to a helmet to be worn by a user, for example by a strap or harness, in addition to or as an alternative to the strap 16.

The mask 14 may be referred to as an oro-nasal mask, for example. In this embodiment the mask 14 is configured to filter ambient gas inhaled into the mask 14 by a person wearing the mask 14, to produce filtered gas for breathing by the person wearing the mask 14. The mask 14 can therefore protect the person wearing the mask from inhaling harmful substances in the ambient gas. In particular, as discussed below, the mask 14 includes one or more filters connected to the mask 14 that include a filtration medium, such as activated carbon, that filters ambient air (or other gas) inhaled into the mask 14 from outside the mask 14 by a person wearing the mask 14, to produce filtered air (or other filtered gas), for breathing by the person wearing the mask 14. Such a mask 14 may be referred to as a filtration mask, for example. In embodiments, the mask 14 may preferably be a half-mask as described below.

In an alternative embodiment, the mask 14 may instead be connected to a source of breathable gas, for example a container or tank of breathable gas, which is typically pressurised (e.g., a container of compressed breathable gas), to supply gas to the mask 14, or an external air filter. The breathable gas may comprise filtered air. The gas in the mask 14 breathed in by the person wearing the mask may therefore be separate to and/or isolated from ambient gas/air on the outside of the mask 14.

The mask 14 is configured to form a seal around the nose and mouth of the person wearing the mask. The mask comprises a seal configured to form the seal around the nose and mouth of the person wearing the mask. For example, the mask 14 may comprise a rigid nose-cup to define a cavity around the nose and mouth of the wearer, with the seal around the periphery of the cavity. The seal may be a substantially air-tight seal.

In this embodiment the mask 14 is held in place over the nose and mouth of the person wearing the mask 14 using a harness 900, described below. In other embodiments, the mask 14 may instead be held in place using one or more straps, for example one or more head straps.

The mask 14 is configured to provide a sealed breathing cavity or space that extends from the top of the nose of the user to a point between the chin of the user and the mouth of the user. The sealed breathing cavity therefore does not include the chin of the user. The mask 14 therefore has a similar geometry to a quarter mask. The sealed breathing cavity or space may be referred to as an oronasal cavity, for example.

The mask 14 may comprise an outlet valve or exhale valve that is configured to allow flow of gas in a direction from the inside of the mask to the surrounding environment and to prevent flow of gas in a direction from the surrounding environment to the inside of the mask. Therefore, air or gas breathed out by a user of the mask 14 can be discharged from the mask 14.

As described in more detail below, the goggles 12 and the mask 14 are provided as separate (i.e., modular) components which can be easily disconnected and connected to one another, and are in particular designed such that they can be easily disconnected and connected to one another while a user is wearing either of the goggles 12 or the mask 14.

As discussed below, the respirator 10 comprises a number of features which provide improved usability and/or performance of the respirator 10, for example improved sealing between the respirator 10 and a wearer's face to provide increased protection against gases, vapours, and/or airborne particles, and/or improved filtration for air inhaled by a user.

Of course, any of the features described below may be provided independently and/or in isolation from the other features described below.

Although the present description relates to a respirator 10 in which the goggles 12 and mask 14 are separate components that can be connected and disconnected from each other, in other embodiments the goggles 12 and mask 14 may be fixed together, for example permanently fixed together, and/or integral. In other words the goggles 12 and mask 14 may not be module and/or detachable in some embodiments.

In addition, other embodiments of the present disclosure may relate to only goggles (i.e. not including the mask) or only a mask (i.e. not including the goggles), instead of a respirator that comprises both goggles and a mask.

The goggles 12 maybe referred to as an upper faceplate section of the respirator 10, and/or the mask 14 may be referred to as a lower faceplate section of the respirator 10.

### Positive Pressure Goggles

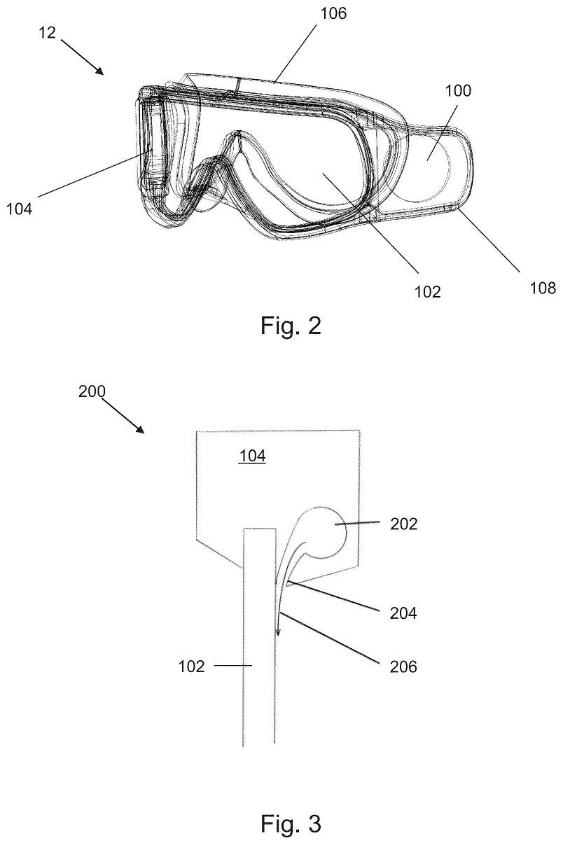

A perspective view of the goggles 12 is shown in FIG. 2. As outlined above, the goggles 12 provide eye protection for a wearer. The goggles 12 comprises a visor 102 (which may also be referred to as an eyepiece), which is made of a transparent material (e.g., polycarbonate or polyurethane) for visibility, and a frame 104 which borders the visor 102 to provide structural rigidity, and also acts as a mounting point or housing for other components of the goggles 12, as described below. For example, the frame 104 may comprise polyamide 66 (PA66, or Nylon 66), polybutylene terephthalate (PBT), or polypropylene.

On a rear side of the frame 104, there is a seal 106, at least a portion of which may be an inflatable seal as described below. The seal 106 is made of a flexible material (e.g., an elastomeric material, such as silicone or butyl rubber formulation), and is configured to provide an airtight (or substantially airtight) barrier between the frame 104 and the face of a wearer when in use. The seal 106 thereby defines a sealed ocular space which is a region between the goggles 12 and around a wearer's eyes, which the goggles 12 keeps free of dangerous gases, vapours, and/or airborne particles.

The lateral edges of the frame 104 extend away from the visor 102 to form projecting wings 108, which may be used as a housing for certain components as described below, and also provide a mounting point for a strap 16 for securing the goggles 12 onto a wearer's head.

The goggles 12 comprises a pump 100, which is located in one of the wings 108 of the frame 104, and is configured to move air and/or gas into the sealed ocular space from the surrounding atmosphere. The pump 100 may be a miniature pump, such as a micropump, for example a piezoelectric micropump or a diaphragm pump, which provides an air flow into the sealed ocular space in order to generate a positive pressure within the sealed ocular space of the goggles 12. The pump 100 may provide an air flow rate of less than 2 litres per minute (lpm), for example, such as around 1 lpm, or more.

An inlet of the pump 100 is in fluid communication, either directly or via one or more flow passages such as a tube or passageway or pipe, with the air outside of the goggles 12. In addition, an outlet of the pump 100 is in fluid communication, either directly or via one or more flow passages such as a tube or passageway or pipe, with air in the sealed ocular space of the goggles 12.

A piezoelectric micropump may operate at a frequency above 20 kHz, i.e., beyond the range of a wearer's hearing, and so act as a silent pump (or substantially silent pump). The pump may pull air into the goggles 12 through a filter to ensure that no contaminants are drawn into the sealed ocular region of the goggles 12. The googles 12 may therefore comprise a filter arranged in an air flow generated by the pump 100 so as to filter the air flow. For example, the filter may be provided over the pump 100 and/or on an external surface of the pump or the goggles 12. The filter may be detachable and/or removable for ease of changing.

A positive pressure means that a pressure inside the sealed ocular space is higher than an ambient pressure of the air surrounding the goggles 12.

The pump 100 may be configured to maintain (or substantially maintain) a predetermined pressure in the sealed ocular space, and/or to maintain (or substantially maintain) a predetermined difference in pressure between the pressure in the sealed ocular space and an ambient pressure in the air surrounding the goggles 12.

The goggles 12 may comprise a pressure sensor for sensing a pressure of the air in the sealed ocular space, and/or a pressure sensor or pressure differential sensor for sensing a difference in pressure between the pressure in the sealed ocular space and an ambient pressure in the air surrounding the goggles 12.

The goggles may comprise a controller that is configured to control an operation of the pump 100 based on (as a function of) an output of the pressure sensor, for example to maintain (or substantially maintain) a predetermined pressure in the sealed ocular space, and/or to maintain (or substantially maintain) a predetermined difference in pressure between the pressure in the sealed ocular space and an ambient pressure in the air surrounding the goggles 12. For example, the controller may be configured to control operation of the pump 100 in order to maintain a positive pressure differential within the ocular space compared with the ambient environment of between 2 and 10 mbar (200 to 1000 Pa). The controller may be located in a wing 108 of the frame 104, and a battery for powering the positive pressure arrangement may be provided in the same wing 108 or the opposite wing. The controller may be implemented as a processing device (e.g. a microcontroller or the like), which is coupled to the pressure sensor to receive an output signal from the pressure sensor, the output signal being indicative of the pressure in the ocular space. The controller may further be coupled to the pump 100 to transmit a control signal to the pump 100 for controlling operation of the pump.

By generating a positive pressure within the goggles 12 in this way, gases, vapours, and/or airborne particles may be prevented from entering the goggles 12 through any regions of the seal 106 which do not form an airtight seal with a user's face.

Although not shown, the goggles 12 comprise an outlet valve (e.g. an exhale valve) elsewhere on the frame 104 to allow air to flow out of sealed ocular region to the surrounding environment and avoid over-pressurising the sealed ocular space which could comprise the efficacy of the goggles 12. The outlet valve is configured to allow flow of gas in a direction from the inside of the sealed ocular region to the surrounding environment and to prevent flow of gas in a direction from the surrounding environment to the inside of the sealed ocular region. The outlet valve may also be tuned or configured to maintain the required pressure differential between the goggles 12 and the ambient atmosphere. For example, the outlet valve may be a pressure relief valve which is configured to only release gas when a threshold pressure is reached within the ocular space.

Air may be input into the sealed ocular space of the goggles 12 using the active defogging arrangement described below. However, this is not essential, and the positive pressure arrangement and the active defogging arrangement may be provided separately and/or independently. For example, the positive pressure arrangement described above and the active defogging arrangement described below may each comprise a separate pump (both pumps drawing air through a single filter, or each pump provided with its own filter, for example). In other examples, the positive pressure arrangement and active defogging arrangement may share a single pump, and an active diverter valve is arranged to direct pumped air to each arrangement. Additionally or alternatively, the positive pressure arrangement and active defogging arrangement may comprise a shared fluidic device which is controllable to direct air to either arrangement depending on a pressure in the ocular space.

### Active Defogging

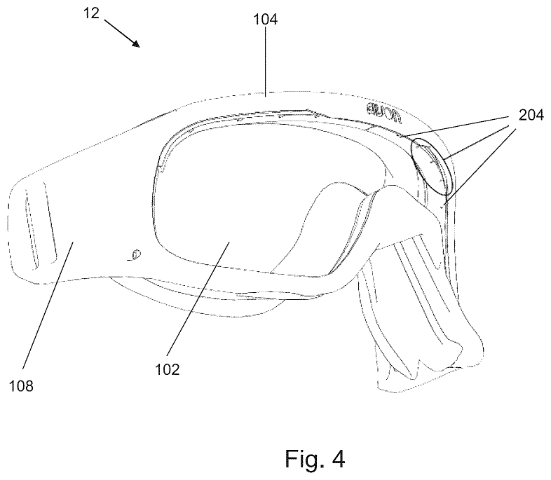

The visor 102 of the goggles 12 is actively defogged by a defogging arrangement 200 which is shown schematically in FIG. 3. A perspective view of the goggles 12 showing the defogging arrangement 200 is shown in FIG. 4.

Fogging of the visor 102 refers to the forming of condensation on the inner surface of the visor 102. For example, the condensation may be caused by vapour due to sweating by the user, and/or vapour in the breath of the user. Such fogging is undesirable since it reduces visibility through the visor 102.

Defogging refers to the prevention or removal or such condensation, so as to maintain or improve visibility though the visor 102.

The defogging arrangement 200 comprises an air channel 202 which runs or extends along at least part of the frame 104, for example along at least an upper portion of the frame 104 that is located at the top of the sealed ocular cavity. A plurality of diffuser holes 204, channels or nozzles extend from the air channel 202 through the frame 104 to diffuser holes 204, which are outlets into the sealed ocular cavity. It will be appreciated that the diffuser holes 204 may be located at any position around the periphery of the visor 102, and advantageously a plurality of diffuser holes 204 may be spaced around the entire periphery of the visor 102 in the frame 104 to ensure that the whole of the visor 102 may be effectively defogged. In certain embodiments, diffuser holes 204 may be provided only along a top edge of the visor 102, allowing a slimmer frame 104 in other regions of the goggles 12 which may be more comfortable for a wearer.

The plurality of diffuser holes 204 are configured (for example angled and/or directed) to direct gas or air from the air channel 202 at and/or across the inside face of the visor. This may aid effective defogging by passing air directly over the inside face of the visor 102. In some examples, the diffuser holes 204 may comprise nozzles which are configured to direct the airflow, and which may also accelerate the air flow to provide an increased airflow velocity to improve the defogging effect (e.g., by defogging the visor more quickly).

In particular, when gas such as air flows through the air channel 202, it is expelled through the diffuser holes 204 and directed across the inside face of the visor 102, as shown by arrow 206 in FIG. 3.

Airflow in the air channel 202 may be generated using a pump, for example a miniature pump, such as a micropump, for example a piezoelectric micropump or diaphragm pump, which may have any of the features of the pump described above. However, in alternative embodiments a different air flow mechanism may be provided for generating air flow in the air channel 202, for example a fan. The pump may provide an air flow rate of at least 0.15 lpm, for example 1 lpm or up to around 2 lpm.

For example, the airflow in the air channel 202 may be generated by the same pump which is used to generate a positive pressure within the goggles 12, as discussed above. For example, the airflow through the defogging arrangement 100 may also generate or provide a positive pressure in the sealed ocular space of the goggles 12. Alternatively, the airflow may be generated by an additional pump (e.g., a second piezoelectric micropump, which may also be located in the wings 108 or elsewhere in the frame 104) which draws air into the channel 202 from outside of the goggles 12, and through a filter. A controller may also be provided within the goggles 12 (e.g., within one of the wings 108) for controlling a pump. A power supply, such as a battery, may also be provided in the frame 104, such as in the same wing 108 as the controller, or within the opposite wing.

Of course, the defogging arrangement may be used in goggles without the positive pressure feature described above, for example in any optically clear face or head-mounted visor, such as goggles, visors, eyepieces, glasses, headsets or other head-mounted apparatus (e.g., for protection, vision enhancement or information display) that do not provide an airtight seal with the face of the user.

### Goggle to Mask Connection

The goggles 12 and the mask 14 are connected to one another via a connector 300 which is configured to allow a user to remove either of the goggles 12 or the mask 14 when the respirator 100 is being worn. In particular, the connector 300 allows the mask 14 to be removed while the goggles 12 is still worn by a user. In addition, the connector may allow the mask 14 to be connected to or disconnected from the goggles 12 while the user is already wearing the goggles 12.

Such an arrangement may allow more flexibility of use and/or ease of mounting the respirator 10 on the head of the user compared to an arrangement in which the mask and goggles are integral or can only be attached or detached before mounting on the head of the user or after removing from the head of the user. By being detachably connectable to one another in this way, the connector 300 allows a mask 14 to be donned without requiring the removal of goggles 12, and the respiratory protection can be achieved without removing other headgear (e.g., helmets, headsets etc.) which are already fitted. In addition, in situations where eye protection (e.g., from projectiles, debris etc.) is required but respiratory protection is not, removing the mask 14 but leaving goggles 12 fitted to reduce the burden may be desirable.

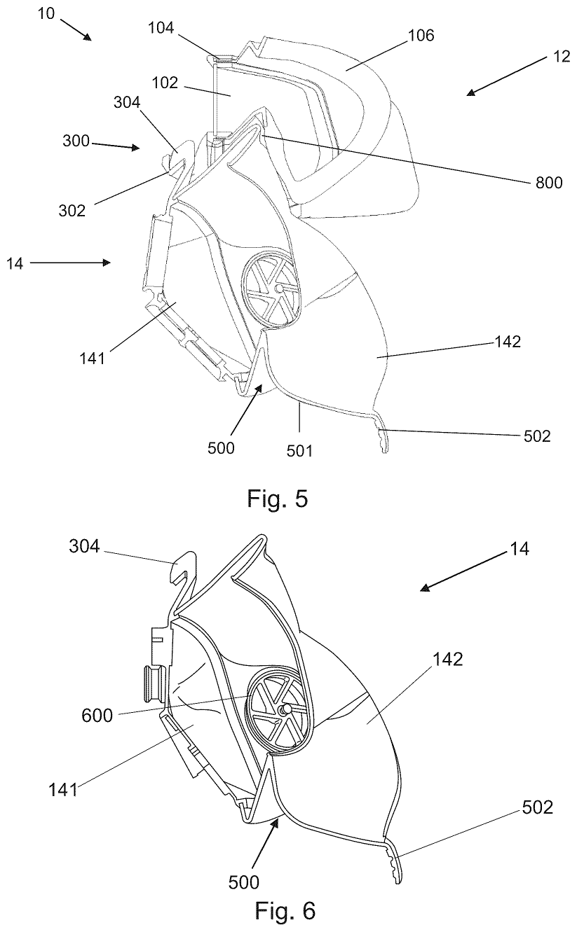

FIG. 5 shows a cross-section view of the respirator 10, and in particular shows the connector 300 when the goggles 12 and the mask 14 are coupled together. FIG. 6 is a cross-section view of the mask 14, separate from the goggles 12.

The mask comprises a nose-cup 141, which is constructed of a rigid plastic to form an enclosed region around a wearer's mouth and nose, and a seal 142 made of a flexible material. The seal 142 is configured to provide an airtight barrier (or substantially airtight barrier) between the nose-cup 141 and the face of a wearer when in use.

The connector 300 is provided in two parts, wherein the goggles 12 comprises a first part 302 of the connector 300 and the mask 14 comprises a second part 304 of the connector 300. The first part 302 is provided in the form of a catch (such as a loop or an eyelet) which is integrated into a portion of the frame 104 which passes over the nose of a user (which may be referred to as a nose bridge of the goggles 12). The second part 304 is provided as a forward-facing hook (i.e., the opening of the hook is arranged to face away from a wearer in use) which is configured to engage with the catch in order to couple the mask 14 to the goggles 12. It will be appreciated that in other embodiments, the first part 302 may be located on the mask 14, and the second part 304 may be located on the goggles 12.

The second part 304 projects upwardly from the nose-cup 141, that is, in a first direction away from the mask. By being provided in this way, the connector 300 allows a wearer to connect the mask 14 to the goggles 12, and the mask 14 is held in place by the connector 300 when a wearer lets go of the mask 14 and uses their hands to attach the mask to their head using a strap or harness and/or to connect a helmet chin strap.

The opening of the hook faces at least partly downwards (i.e., in a direction at least partly opposite to the first direction), so that, when the mask 14 is connected to the goggles 12, the mask 14 cannot be pulled downwards away from the goggles without lifting and/or rotating the mask 14 to disconnect the mask 14 from the goggles 12. By being shaped in this way, the hook also ensures that the mask 14 may hang from the goggles 12 when attached, and also ensures fitment can only occur in one direction—that is, from beneath the goggles 12, between the wearer's nose and the goggles 12.

In addition, as the connector 300 is configured generally as a loop 302 and hook 304 arrangement, the connector 300 provides a degree of independent movement between the goggles 12 and the mask 14, which allows these parts to each retain a good seal with the face of a wearer in use.

The first part 302 and/or the second part 304 may be configured (for example shaped and/or sized and/or positioned) so that the first part can only be inserted into or removed from the second part when the mask is tilted or angled relative to a configuration of the mask when the mask is attached to the face of the user.

The first part 302 and/or the second part 304 may comprise a magnet for increasing a connection strength between the first part 302 and the second part 304 when they are connected together. A magnetic connector may also be used to provide a detachable connection in addition to the connector 300. Use of magnets in this way may also assist the fitting processing.

### Close-Coupled Seal

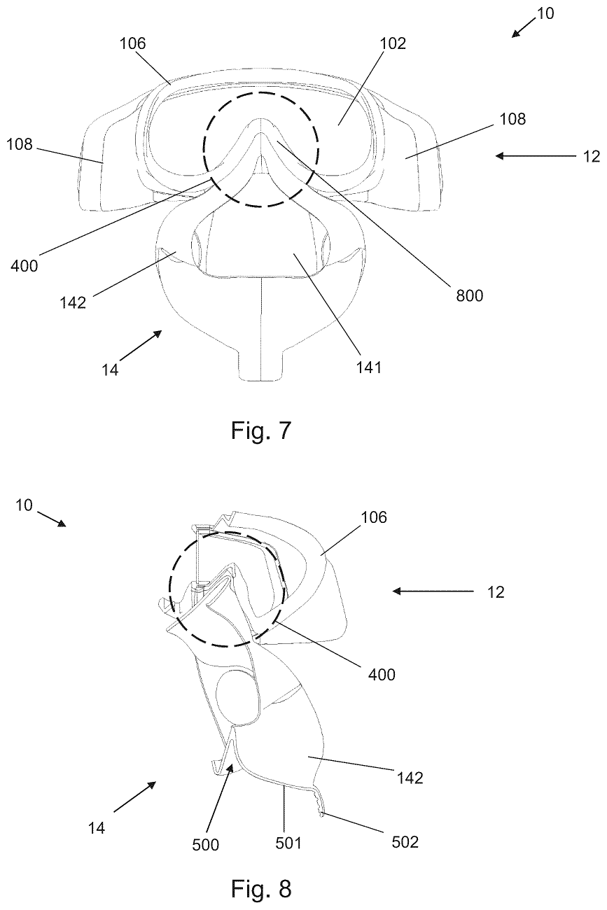

FIG. 7 shows a rear view of the respirator 10, with the goggles 12 coupled to the mask 14. As highlighted by a circle, at a portion of the seal 106 of the goggles 12 (which may have any of the features of the seal 106 described above) and the seal 142 of the mask 14 (which may have any of the features of the seal 142 described above) which is configured to pass over the bridge of a wearer's nose (the bridge region 400), the seals 106, 142 are configured to have complementary, or tessellating, or congruous, shapes such that there is no gap (or substantially no gap) between the goggles 12 and the mask 14. For example, the seals 106, 142 may have tangential or colinear surfaces to one another. There may be a continuous curve between the two seals. The two seals may form a continuous surface or a continuous line between them. There may be an airtight seal between the two seals.

By being shaped in this way, the risk of leakage at the interface between the seals 106, 142 is reduced, and the goggles 12 and the mask 14 do not compromise the seal of the other part. In addition, the seals 106, 142 are shaped in this region to provide a large contact area with the face of a wearer in order to ensure that the regions enclosed by the goggles 12 and the mask 14 are airtight (or substantially airtight) at all times.

As shown in more detail in FIG. 8, which is a cross-section view of the respirator 10, the seal 106 of the goggles 12 and the seal 142 of the mask 14 overlap one another in the bridge region 400. In this way, the seals 106, 142 form a layered structure in the bridge region 400, without leak paths between the seals 106, 142, which provides additional protection to ensure that the areas enclosed by the goggles 12 and the mask 14 are airtight to prevent ingress of contaminants such as unwanted or harmful gases, vapours and/or particles. In particular, the seals 106, 142 may form complementing reflex seals to prevent ingress of contaminants. The seals may be made of an elastomeric material, or foam for example. As can be seen in FIG. 7, the seal 106 of the goggles 12 and the seal 142 of the mask overlap one another to form a continuous sealing surface which is arranged to extend around (or over) a bridge of the wearer's nose, such that the continuous sealing surface extends from a left side of the wearer's nose to a right side of the wearer's nose.

### Nose Cup Chin Strap

Respirator masks are typically available in three variants, which are known as quarter-mask, half-mask or full-face, with a full-face mask typically providing the highest level of protection and a quarter-mask the least (based on the reliability of a seal which is formed between the mask and a wearer's face). A quarter-mask is configured to fit between a wearer between the top of the nose and the top of the chin, which makes a quarter-mask relatively easy to dislodge compared with other variants. A half-mask is larger and more difficult to dislodge, being configured to fit between the top of the nose and under the chin, thereby providing a generally higher level of protection. However, fitting in this way means that a half-mask is difficult to wear with a helmet, as the chinstrap of the helmet can interfere with the seal of the half-mask. By way of comparison, a full-face mask is configured to fit from a wearer's hairline to under the chin. The respirator 10 according to the present disclosure provides full-face protection by way of two separate components, the goggles 12 and the mask 14.

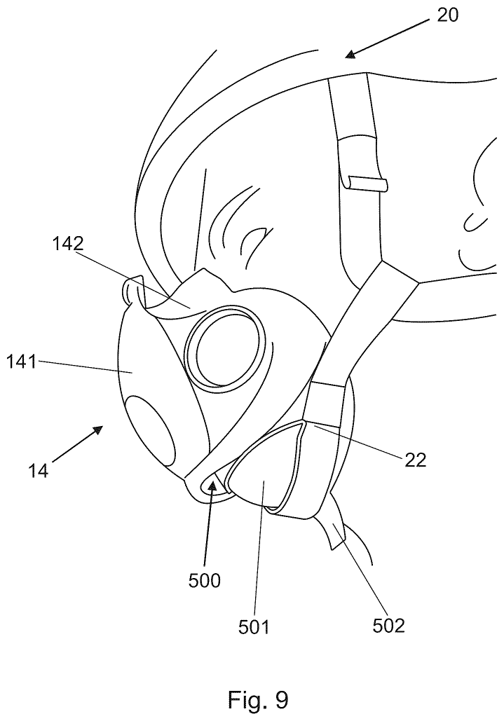

FIG. 9 shows a perspective view of a user wearing a mask 14 as a standalone mask (i.e., without goggles 12), with a helmet 20. Of course, in practice the mask 14 can be combined with the goggles 12 as discussed above. The mask 14 is configured such that the nose-cup 141 has a similar geometry to a quarter-mask. In particular, the mask 14 is configured to provide a sealed breathing cavity or space that extends from the nose of the user (for example the top of the nose of the user) to a point between the chin of the user and the mouth of the user. The sealed breathing cavity therefore does not include the chin of the user and/or does not extend to a bottom of the chin of the user. The sealed breathing cavity or space may be referred to as an oronasal cavity, for example.

However, the seal of the mask 14 includes a chin portion 501 that is configured to extend over the chin of the user wearing the mask 14. The chin portion 501 is shaped to fit over the chin of a user and to conform to the shape of the chin of the user, and forms a continuous seal with the rest of the seal 142, such that the sealing surface of the mask 14 is continuous around and through the chin portion 501 and the remainder of the seal 142. In particular, an external shape of the chin portion 501 is configured to correspond to, or conform to, the shape of the chin of the user. Therefore, when the user is wearing the mask 14, a shape of the chin portion 501 of the seal of the mask 14 is substantially the same as a shape of the user's chin. This means that the mask 14 can be worn with a helmet 20 with a standard chin strap 22, since the chin strap 22 of the helmet 20 can be fitted over the chin portion 501 of the seal of the mask 14 in the same way as it can be fitted over the chin of a user.

In addition, the chin portion 501 being shaped to fit over the chin of the user may facilitate correctly locating the mask on the face of the user and/or maintain the mask in a correct position on the face of the user in use. The chinstrap also applies pressure on the seal 142 at the chin portion 501 to push against the wearer's chin, improving the seal.

The chin portion 501 of the seal may comprise a sheet or layer of material, for example a sheet of an elastomeric material such as silicone or butyl rubber formulation.

The seal 142 comprises a folded region 500, which is also shown in FIGS. 4, 5 and 7, the folded region 500 being located where the seal 142 meets the nose-cup 141. The folded region 500 forms a collapsible and/or deformable area which allows the chin portion 501 to move relative to the nose cup without compromising the seal. The folded region 500 forms a pleat which can receive a chinstrap 22 when the mask 14 is worn by a user. In this way, the folded region 500 allows a chinstrap 22 to pass directly over the chin portion 501 of the seal 142, between the bottom of the chin of the user and the mouth of the user, without the need for helmet adaptor straps or a reduction in potential respiratory protection (e.g., which may otherwise occur as a result of the chinstrap 22 disturbing the chin portion of the seal 142). The seal 142 of the mask 14 thereby provides an increased level of protection when compared with typical quarter-masks or half-masks. As shown in FIG. 8, the folded region 500 is arranged such that the pleat is located at the front of a wearer's chin, underneath the nose-cup 141, to allow a chinstrap 22 to be positioned directly over the top of the chin portion 501 of the seal 142. The pleat also allows movement of the chin (e.g., when talking) without stretching the seal 142 in an undesirable way, which could cause leak paths. The folded region 500 thereby makes the mask 14 more compliant to a wearer's facial movements. In other examples, the folded region 500 may comprise a bellows, or other deformable feature, to provide such effects.

The chin portion 501 also includes a tab 502, which is a protrusion from the chin portion 501, for the wearer the hold the mask 14 in the correct position while they pull and tighten the chinstrap 22 over the mask 14 and the seal 142. This may ensure that the chin portion 501 is correctly located when the helmet 20 is being fitted.

### Single Action Filter Mount Connector

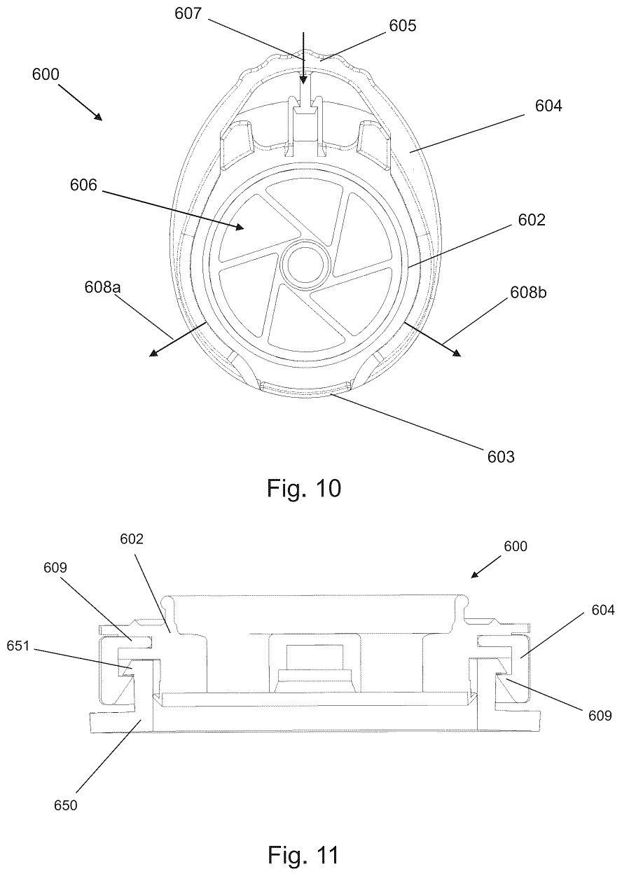

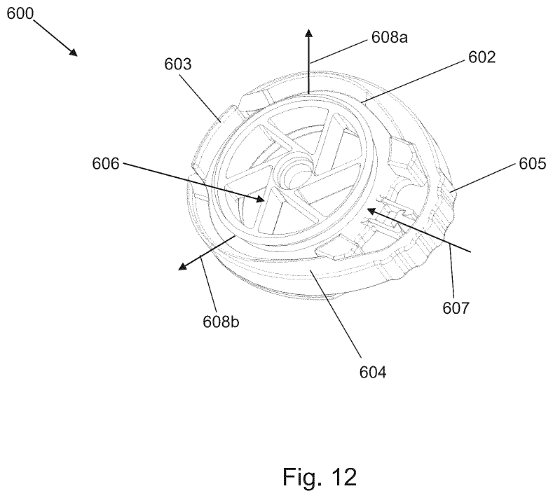

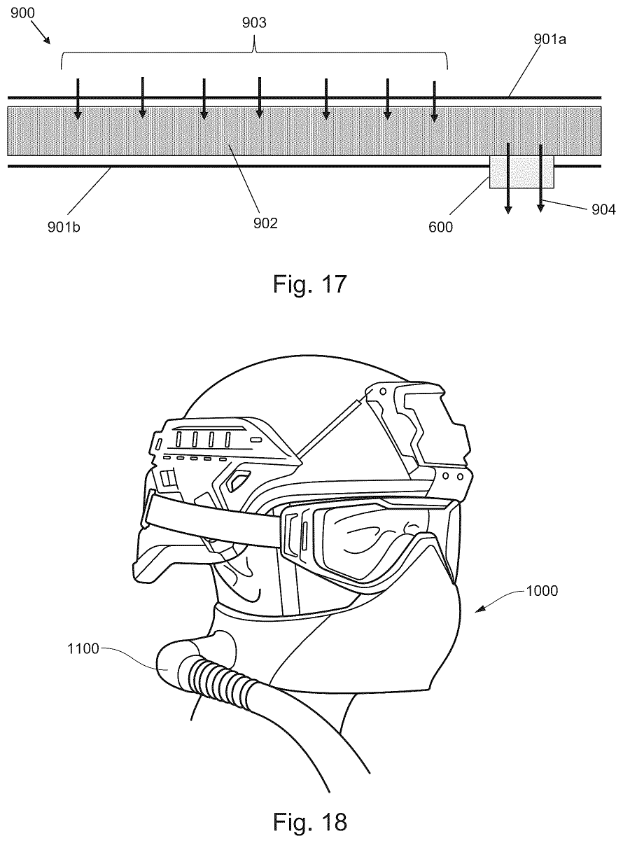

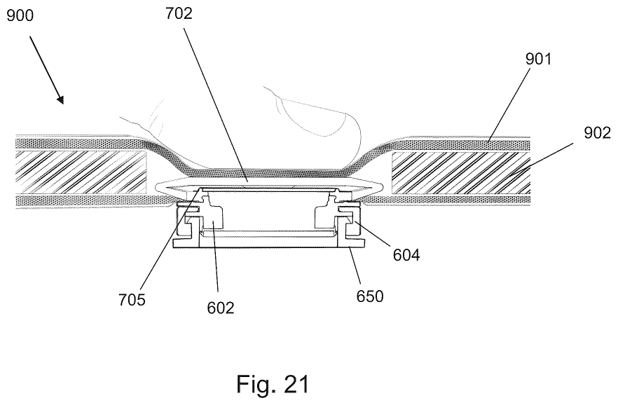

FIG. 10 shows a bottom view of a clip 600 for mounting a filter onto a filter mount of a respirator (e.g., a respirator 10 as described herein). FIG. 12 shows a perspective view of the clip 600. FIG. 20 shows a cross section view of a harness 900 as described below with respect to FIG. 17, which is configured to also provide a filter, wherein the harness 900 includes a filter clip 600 and a negative pressure test valve 700, wherein the valve 700 is in an open position. The filter clip 600 is connected to a filter mount 650 of a mask. FIG. 21 shows a similar arrangement, where the valve 700 is in a closed position.

In particular, the clip 600 in FIG. 10 is for mounting a filter onto a filter mount of the mask 14 of the respirator 10 described above.

As mentioned above, the mask 14 includes one or more filters connected to the mask 14 that include a filtration medium, such as activated carbon, that filters ambient gas inhaled into the mask 14 from outside the mask 14 by a person wearing the mask 14, to produce filtered gas for breathing by the person wearing the mask 14. Such a mask 14 may be referred to as a filtration mask, for example.

Such filters are typically removable and/or detachable from the mask 14, so that the filters can be replaced, for example after a predetermined period of time.

In this embodiment, a filter is detachably attached to the mask 14 using the clip 600 for mounting the filter onto a filter mount of the mask 14.

FIG. 11 shows a cross-section view of the clip 600 mounted onto a filter mount 650. The filter mount 650 is fixed to the mask 14, and/or integral with the mask 14. The clip 600 allows a filter to be easily connected to and disconnected from the filter mount 650. In particular, the clip 600 provides a push-to-fit, or snap-fit, mechanism for connecting a filter, and a simple squeeze mechanism to disconnect the filter—movements which are easy to perform using one hand and/or when wearing gloves, for example.

The clip 600 comprises a generally circular main body 602, which defines a central open area 606 for allowing or providing an air flow through the clip 600. Filter material may be received in and/or attached to the open area 606, so that air can be drawing through the filter material into or through the central open area 606.

The main body 602 is made of a rigid material, such as a plastic material, to provide structural strength. In some embodiments, the main body 602 may also house a magnet which can provide a magnetic connection with a corresponding magnet on a filter mount, such as filter mount 650.

Around a majority portion of the circumference of the main body 602, a locking collar 604 is provided. The locking collar 604 is a length of a resiliently deformable material which is connected at each of its ends to an arcuate section 603 at an outer edge of the main body 602, with the length of the locking collar 604 passing around the main body 602. In this way, the locking collar 604 is secured to the main body 602. The locking collar 604 also comprises a flange portion 609 which is configured to be received within a circumferential groove of the main body 602, as shown in FIG. 11, to ensure that airflow through the clip 600 and the filter mount 650 passes through the open area 606 and the filter material contained therein.

The filter mount 650 comprises a generally circular upstanding member 651, which has an outwardly projecting rim along its upper edge. The projecting rim is arranged to engage with an inwardly projecting lip 609 of the locking collar 604 so as to lock the clip 600 onto the filter mount 650.

The inwardly projecting lip 609 is provided on at least part of the locking collar 604, for example on at least two portions of the locking collar 604 on opposite (or substantially opposite) sides of the locking collar 604.

The locking collar 604 is configured such that it takes a generally oval or egg-shaped shape at rest on the main body 602. As a result, a head portion 605 of the locking collar 604 is biased so as to be spaced radially away from the main body 602, with the remainder of the locking collar 604 in close contact with the main body 602. When the clip 600 is mounted on the filter mount 650, the locking collar 604 remains engaged with the upstanding member 651 as described above.

Pushing the head portion 605 radially inwards towards the centre of the main body 602 (in a direction shown by arrow 607), for example by appropriately squeezing the locking collar 604, causes the locking collar 604 to slacken, and splay outwards of the main body 602 (e.g., in directions shown by arrows 608a, 608b). Pressing the head portion 605 radially inwards in this main thereby allows the clip 600 to be removed from a filter mount 650, by disengaging the inwardly projecting lip 609 of the locking collar 604 from the upstanding member 651. In particular, splaying outwards of the locking collar 604 causes the inwardly projecting lip 609 of the locking collar 604 to move outwards so that it is no longer engaged with the upstanding member 651.

The clip 600 can then be lifted away from the filter mount. When the force is removed, the locking collar 604 returns to its original shape due to the elasticity of the material from which it is made. The elasticity of the locking collar 604 also allows the clip 600 to be connected to the filter mount 650 by simply pushing the two parts together.

As shown in FIG. 11, the outwardly projecting rim of the circular upstanding member 651 has a downwardly angled and/or chamfered portion at its distal end. When the clip 600 is pushed towards the filter mount 650, the inwardly projecting lip 609 of the locking collar 604 contacts the downwardly angled and/or chamfered portion of the outwardly projecting rim of the circular upstanding member 651. Continued pressure applied to the clip 600 causes the inwardly projecting lip 609 to be displaced outwards by the downwardly angled and/or chamfered portion of the outwardly projecting rim, causing the locking collar 604 to splay outwards.

Once the inwardly projecting lip 609 of the locking collar 604 has passed the outwardly projecting rim of the circular upstanding member 651, the inwardly projecting lip will move back inwards, so that the locking collar 604 is no longer splayed.

In this arrangement, movement of the clip 600 away from the filter mount 650 is prevented by abutment between an upper surface of the inwardly projecting lip 609 of the locking collar 604 and a lower surface of the outwardly projecting rim of the circular upstanding member 651.

Therefore, the clip 600 can be push-fitted onto the filter mount 650 in a single motion as described above but can only be removed from the filter mount 650 by pushing the head portion 605 as described above.

The clip 600 may be attached to, or fixed to, or integral with, a filter and/or a body comprising or containing filter material.

Of course, in another embodiment the clip 600 may be attached to, or fixed to, or integral with the mask 12 and the filter may comprise the generally circular upstanding member 651 described above.

### Negative Pressure Test Valve

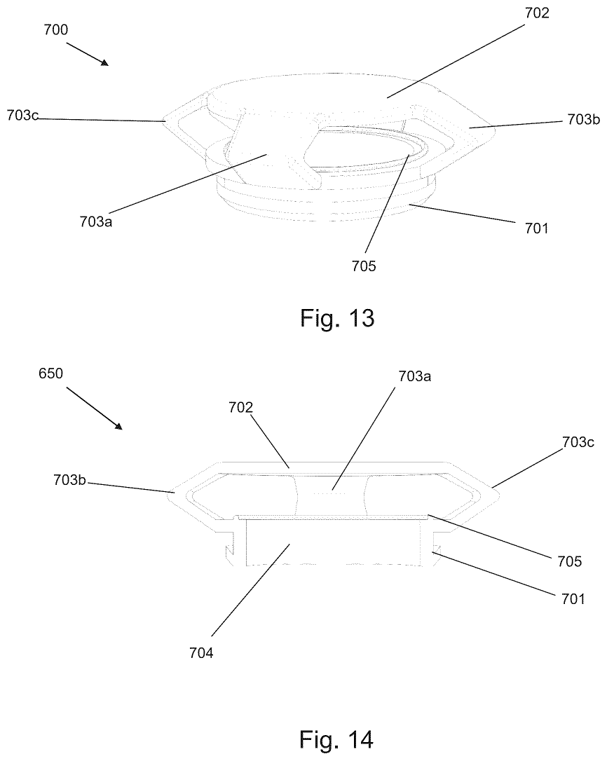

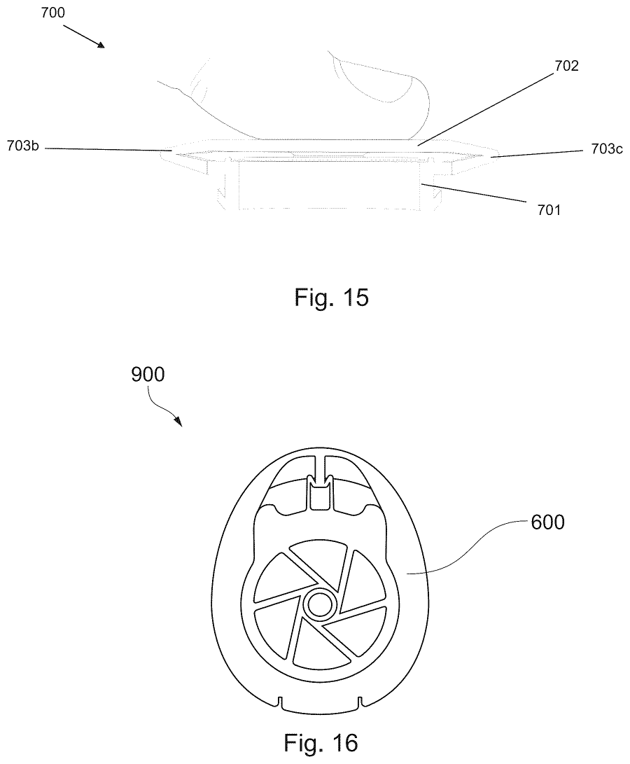

FIG. 13 shows a perspective view, and FIG. 14 shows a cross-section view, of a negative pressure test valve 700 which may be used in embodiments of the present disclosure, in an open position which allows air flow through the valve 700. FIG. 15 shows a cross-section view of the negative pressure test valve 700 in a closed position, being pressed by a user, such that no air can flow through the device. FIG. 20 shows a cross section view of a harness 900 as described below with respect to FIG. 17, which is configured to also provide a filter, wherein the harness 900 includes a filter clip 600 and a negative pressure test valve 700, wherein the valve 700 is in an open position. The filter clip 600 is connected to a filter mount 650 of a mask. FIG. 21 shows a similar arrangement, where the valve 700 is in a closed position.

In order to check that a respirator is properly sealed against the face or head of a user when worn, a user typically needs to cover the filter media with their hands to form an airtight seal. The wearer then inhales to see if there are any breaks in the seal between the respirator and their face. However, it can be difficult to ensure that the filter media is properly covered when testing in this way, which may lead a wearer to incorrectly conclude that a seal is not properly formed when they have simply not fully covered the filter media. The negative pressure test valve 700 provides a mechanism to more easily perform this check.

The pressure test valve 700 may be connected to, or attached to, or integral with (e.g., built into), the filter clip 600 described above, or another type of filter connector, and is operable to selectively and/or temporarily block flow of air through the filter connector. The pressure test valve 700 therefore achieves a similar effect to covering the filter media, but by blocking air flow thorough the filter connector instead.

The negative pressure test valve 700 comprises a base section 701 which is generally circular, and configured to connect to (e.g., via an interference fit) a filter (e.g., over a filter clip 600 as described above).

The base section 701 defines a central aperture 704 to allow airflow through the valve 700 when in the open position as shown in FIGS. 11 and 12. Around an upper surface of the base section 701 a valve seat 705 is provided, which ensures good contact with a cover 702 when the valve 700 is in the closed position as described below.

A cover 702 is spaced apart from the base section 701 by a plurality of biasing elements 703a, 703b, 703c. The biasing elements 70a, 703b, 703c are spaced apart around the circumference of the base section 701 and the cover 702 so that air can flow between them and through the aperture 704 when the valve 700 is in the open position as shown in FIGS. 11, 12 and 20. In order to close the valve 700, a user presses the cover 702 towards the base section 701 to form a seal between the cover and the base section 701. In particular, the cover 702 is pressed against the valve seat 705 which protrudes about the base section 701 to ensure a tight seal. The cover 702 is at least equal in size to the base section 701. By pressing the valve 700 closed in this way, as shown in FIGS. 14 and 21, no air can flow through the aperture 704 and into a respirator, and so a wearer can perform a negative pressure test (e.g., by inhaling when the valve 700 is closed to check for any breaks in the seal between the respirator and their face). When the user stops pressing on the cover 702, the valve 700 returns to the open configuration as the biasing members 703a, 703b, 703c urge the cover 702 away from the base section 702.

As shown in FIGS. 11-13, the valve 700 may be formed as a unitary component from a resiliently deformable material, such as a plastic material, such that the biasing elements 703a, 703b, 703c are formed as a living hinge. However, it will be appreciated that, in other examples, the biasing elements 703a, 703b, 703c may be provided as separate components, for examples springs in addition to or in place of living hinge elements. It will also be appreciated that any number of biasing elements 703a, 703b, 703c may be used as appropriate.

The valve 700 may be a one-piece, unitary, or integral, or single-part component, for example.

### Inflating Seal

It is known that it is difficult to provide and maintain an effective seal around the entire periphery of a goggles portion of a respirator, and/or around an entire periphery of a mask portion of a respirator. For example, it is particularly difficult to seal the region around the bridge of a wearer's nose.

In order to address this issue, at least a portion of the seal 106 of the goggles 12 of the respirator 10, in embodiments of the present disclosure, may be inflatable to change and/or improve the seal between the goggles and the face of the user. For example, at least a portion of the seal 106 of the goggles 12 may comprise one or more inflatable bladders, or bags, or bellows.

For example, a bladder, bag or bellows may be provided in the bridge region 800 as indicated in FIGS. 1, 4 and 6. Of course, it will be appreciated that a plurality of bladders may be present at any suitable region of the seal 106 in order to ensure a good fit. In particular, the one or more bladders, bag or bellows may be inflated when the goggles 12 is worn by a user, and the volume of air within each bladder, bag or bellows may be freely adjustable to ensure a tight seal at all times.

To inflate the bladder, bag or bellows a pump, such as a micropump may be used, such as a piezoelectric micropump, which may be located in the wings 108 of the goggles 12 or in a nose cup of the mask, for example, to pump air into the bladder, bag or bellows. For example, air may be drawn through a filter (e.g., a filter of the mask 14 or a filter in the wings 108 of the goggles 12). In some embodiments, air for inflating the bladder, bag, or bellows may be air which is redirected or recycled from a positive pressure arrangement and/or a defogging arrangement as described above. It may be advantageous that the air used to inflate the bladder, bag, or bellows is filtered air, for example to reduce the risk of compromising the respirator in event of a puncture or tear. In some examples, the pump may use a one-way valve or be otherwise configured to not allow air backflow through the pump (e.g., a diaphragm pump design) in order to ensure that the bladder, bag or bellows may remain inflated in the event of power loss.

The bladder, bag or bellows may also comprise a controllable valve or seal which can be used to release air and deflate the bladder, bag or bellows if required.

A tight seal may be ensured by providing a pressure sensor within each bladder, which can provide feedback to a controller such as a microcontroller, which may be located in the wings 108 of the goggles 12 or in a nose cup of the mask, for example. For example, the bladder may be inflated to a pressure between 1-100 mbar, and the controller may ensure that the desired pressure is maintained. The controller may control the pump and/or valve in order to ensure that the faceplate 12 is properly sealed at all times. The pump and/or valve may also be controllable directly by a user, for example by using buttons on the goggles 12.

The controller may be configured to inflate, or further inflate, the bladder, bag or bellows when a user wearing the goggles 12 provides an instruction to the controller, for example by pressing a button on the goggles 12. This may therefore improve a seal between the goggles and the user when required, for example when the user is exposed to a dangerous environment.

The goggles 12 may comprise a pressure sensor for sensing a pressure of the air inside the goggles, for example in a sealed ocular space of the goggles 12. The goggles may further comprise a controller such as a microcontroller that is configured to operate the pump to inflate the bladder, bag or bellows if the pressure of the air inside the goggles is below a predetermined threshold.

The goggles 12 may comprise a differential pressure sensor for sensing a pressure difference between a pressure of the air inside the goggles 12, for example in a sealed ocular space of the goggles 12, and the pressure of air outside the goggles 12. The goggles may further comprise a controller such as a microcontroller that is configured to operate the pump to inflate the bladder, bag or bellows if the pressure different is below a predetermined threshold.

In addition, or alternatively, the goggles 12 may comprise an inflatable part, such as an inflatable bladder, bag or bellows that is inflatable so as to push a strap of the goggles outwards to tighten a fit of the goggles 12 to the user, so as to improve a seal between the goggles 12 and the face of the user. Such a bladder, bag or bellows may be controlled by a controller as in any of the examples described above, for example.

In addition, or alternatively, at least a portion of a seal 142 of the mask 14 in embodiments of the present disclosure may be inflatable in any of the ways described above to change and/or improve the seal between the goggles and the face of the user, which may be controllable in any of the ways described above.

In addition, or alternatively, the mask 14 may comprise an inflatable part, such as an inflatable bladder, bag or bellows that is inflatable so as to push a strap or harness of the mask 14 outwards to tighten a fit of the mask 14 to the user, so as to improve a seal between the mask 14 and the face of the user. Such a bladder, bag or bellows may be controlled by a controller as in any of the examples described above, for example.

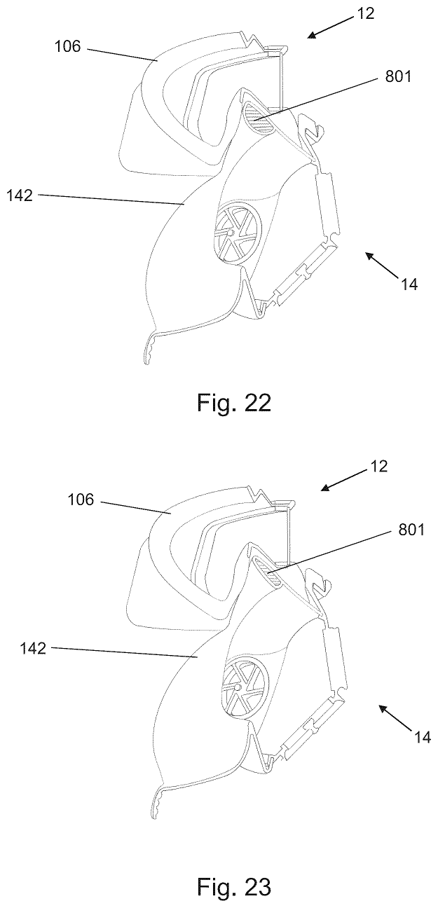

FIG. 22 shows a cross-section view of a respirator comprising an inflatable bladder 801 in a nasal bridge portion of the seal 142 of a mask 14, wherein the inflatable bladder 801 is inflated in order to provide an improved fit between the seal 142 and a wearer's face. FIG. 23 shows a cross-section view of the respirator comprising the inflatable bladder 801, wherein the inflatable bladder 801 is deflated, and so the fit between the seal 142 and a wearer's face may be looser (e.g., for increased comfort, or in a less hazardous environment).

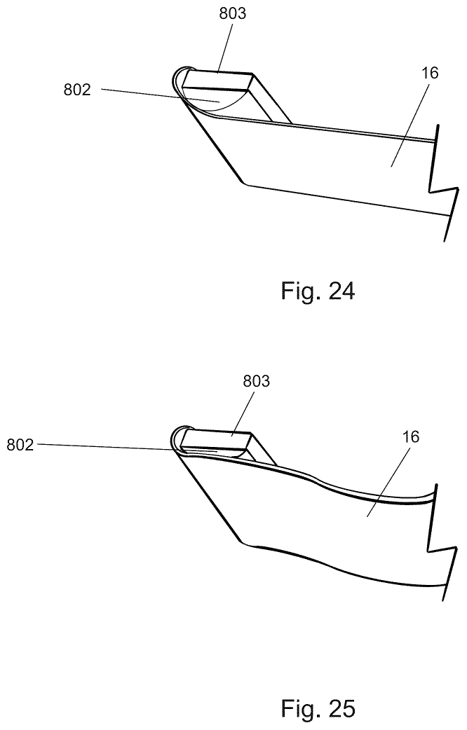

FIG. 24 shows an inflatable bladder 802 forming part of a strap 16 for a respirator (e.g., a strap for goggles 12 or mask 14) when the inflatable bladder 802 is inflated, and so acting to tighten the strap 16, in order to improve a seal between the respirator and the wearer's face. FIG. 25 shows the inflatable bladder 802 in a deflated state, which provides slack in the strap 16 (e.g., for increased comfort, or in a less hazardous environment). The inflatable bladder 802 is mounted on a rigid base 803. The rigid base 803 may house other components, such as a pump or a fan for inflating the bladder 802, and a controller for controlling operation of the pump or fan. The inflatable bladder 802 is arranged on a rear side of the strap 16, such that the rigid base 803 is closer to a wearer's head, in order to provide outward pressure on the strap 16 when the bladder 802 is inflated, thereby tautening the strap 16 to provide an improved seal between the respirator (e.g., either or both of goggles 12 or a mask 14) and the face of the wearer.

It will be appreciated that such inflatable bladder arrangements may be used with any device which comprises a face seal, or sealing arrangement configured to seal against a user's face.

### Disposable Filter in Mask Harness

In embodiments of the present disclosure, the mask headband, or neckband, or retainer, or harness, or mounting arrangement, which holds the mask in place on a wearer, may be used to provide a disposable filter. For example, as shown in FIG. 1, the mask 14 is secured to a wearer by a harness 900 which passes from the front of the mask 14 to a rear of the wearer's head, where the two ends of the harness 900 may be secured to one another, e.g., with a Velcro™ attachment or a hook and loop fastener. By distributing the disposable filter over a large area in this way, breathing resistance of the filter is reduced compared with conventional filters.

FIG. 16 shows a rear view of the harness 900, showing that the harness 900 comprises a filter clip 600 on its inner surface for connecting to a filter mount 650 of the mask 14. It will be appreciated that, in some embodiments, the harness 900 may comprise more than one such filter clip 600 arranged to connect with respective filter mounts 650 of the mask 14. The filter clip 600 may be the same as that described above, or another type of filter clip.

FIG. 17 shows a schematic drawing of the harness 900. The outer surface of the harness 900 comprises a filter 901, which comprises a filter material (such as a fluroplastic porous film, electret, spunbond or meltblown materials, or any combination of such materials), and may further comprise a cover material (such as a breathable fabric) over the filter material. The filter 901 is provided as an outer layer 901a and an inner layer 901b, which are joined together at the edges of the harness 900, or may be provided as a single sheet or material. In some examples, the cover material may be printed with a pattern, such as a camouflage pattern. The filter 901 may be pleated, folded, or creased (e.g., by thermoforming) so as to maximise the surface area of the harness 900 and so decrease breathing resistance. In some examples, the filter 901 may cover only a portion of the harness 900, and in particular a region of the harness 900 which is located on and around a user's neck in use may comprise a highly breathable fabric to reduce the heat burden of the harness.

The filter 901 is configured to filter gas such as air that is breathed into the mask via the harness 900.

Between the outer layer 901a and the inner layer 901b of the filter 901, the harness 900 comprises a plenum space 902. The plenum 902 is an enclosed area, or space, or volume in the harness 900 through which air can flow. The plenum space 902 comprises a spacer material, or a flexible air permeable structure, to allow airflow through the plenum space 902 and to the filter clip 600, where air is introduced into a mask 14. The material which fills the plenum space 902 should preferably be a resilient material to ensure that airflow cannot be easily cut off. For example, the spacer material may be a 3D spacer fabric, expanded paper, an open cell foam, or a 3D printed lattice (e.g., plastic chainmail). Any suitable material may be used to fill the plenum space 902 to increase the breathability through the harness 900, and should preferably be flexible for increased comfort of the harness 900.

The harness 900 may therefore comprise a laminate structure comprising a 3D spacer material sandwiched between the inner layer 901b and the outer layer 901a of the filter 901. For example, the 3D spacer material may be a 3D spacer fabric, expanded paper, an open cell foam, or a 3D printed lattice.

As mentioned above, the inner layer 901b and outer layer 901a of the filter 901 are joined together at the edges of the harness. Therefore, gas such as air may only be able to enter the plenum through the inner layer 901b and outer layer 901a of the filter.

Of course, in other embodiments the inner layer 901a may comprise a gas impermeable membrane or material instead of the filter 901, so that gas such as air can only enter the plenum through the outer layer 901a of the filter 901. In this case, the gas impermeable membrane or material is omitted where the filter clip 600 is provides, so that gas such as air can flow out from the plenum through the filter clip 600.

When a wearer breathes, air is drawn into the harness 900 through the filter 901 (for example at least through the outer layer 901a of the filter 901), as shown by arrows 903. The filter material 901 thereby filters the air, removing any potential contaminants. The filtered air then passes through the plenum space 902, and through the filter clip 600, as shown by arrows 904, where it passes into the mask 14 worn by a user, where it can be safely inhaled.

FIG. 20 shows a cross section view of the harness 900, wherein the harness 900 includes a filter clip 600 and a negative pressure test valve 700, wherein the valve 700 is in an open position. The filter clip 600 is connected to a filter mount 650 of a mask. FIG. 21 shows a similar arrangement, where the valve 700 is in a closed position.

### Mask Harness as External Air Source Connection

In embodiments of the present disclosure, the mask headband, neckband, or harness, which holds the mask in place on a wearer, may provide a connection point to allow the transfer of purified air from an external filter or air source (e.g., a remote-mounted filter or PAPR) into the mask of the respirator. For example, such a harness 1000 may be used as an alternative to the harness described above with respect to FIG. 17. FIG. 18 shows an example of a harness 1000 which is used to connect a mask (mask portion) to an external air supply or external filter via a hose 1100. The mask is secured to a wearer by the harness 1000 which passes from the front of the mask to a rear of the wearer's head, where the two ends of the harness 1000 may be secured to one another, e.g., with a Velcro™ attachment or a hook and loop fastener.

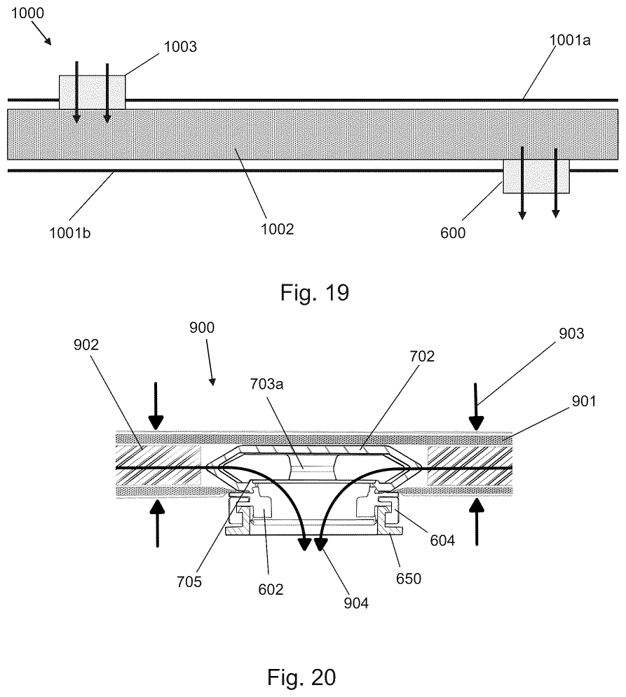

A schematic diagram of the harness 1000 is shown in FIG. 19. The outer surface of the harness 1000 comprises a gas and liquid impermeable membrane 1001, which forms a barrier protecting against potential CBRN contaminants. For example, the membrane 1001 may be formed of polyurethane, or butyl rubber. The membrane 1001 is provided as an outer layer 1001a and an inner layer 1001b, which are sealed together at the edges of the harness 1000. In some examples, the membrane 1001 may be printed with a pattern, such as a camouflage pattern. Air is introduced through the harness 1000 at an input port 1003, which may be configured to connect to a hose for receiving filtered air or other external air or gas, e.g., from an external filter or an air or gas supply, for example such as hose 1100 as shown in FIG. 18. In particular, the gas and liquid impermeable membrane 1001 is omitted where the input port 1003 is provided so that gas can be input to the harness 1000 thorough the input port 1003.

Between the outer layer 1001a and the inner layer 1001b of the membrane, the harness 1000 comprises a plenum space 1002. The plenum 1002 is an enclosed area, or space, or volume in the harness 900 through which air can flow. The plenum space 1002 comprises a spacer material, or flexible air permeable structure, to allow airflow through the plenum space 1002 and to a filter clip 600, where air is introduced into a mask. The filter clip may be as illustrated in FIG. 15 and described above, for example. The gas and liquid impermeable membrane 1001 is omitted where the filter clip 600 is provided so that gas can be output from the harness 1000 through the filter clip 600.

The material which fills the plenum space 1002 should preferably be a resilient material to ensure that airflow cannot be easily cut off. For example, the spacer material may be a 3D spacer fabric, expanded paper, an open cell foam, or a 3D printed lattice (e.g., plastic chainmail). Any suitable material may be used to fill the plenum space 1002 to increase the breathability through the harness 1000, and should preferably be flexible for increased comfort of the harness 1000.

When a wearer breathes, air is drawn into the harness 1000 from an external source through the input port 1003, for example via a valve in the mask 14 and/or in the hose 1100 and/or in the external source. The pre-filtered air and/or external air or gas from the external source then passes through the plenum space 1002, and through the filter clip 600, where it passes into the mask worn by a user, where it can be safely inhaled.

The mask 14 further comprise an outlet valve, for example an exhale valve, for allowing air and/or gas exhaled by the user to exit from the mask 14.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the disclosure in diverse forms thereof.

While the disclosure has been described in conjunction with the exemplary embodiments described above, many equivalent modifications and variations will be apparent to those skilled in the art when given this disclosure. Accordingly, the exemplary embodiments of the disclosure set forth above are considered to be illustrative and not limiting. Various changes to the described embodiments may be made without departing from the spirit and scope of the disclosure.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Additional aspects and/or embodiments of the present disclosure are set out in the following sets of numbered clauses. The section headings below are for organizational purposes only and are not to be construed as limiting the subject matter described.

### 1. Positive Pressure Goggles

1. Protective goggles comprising:
   a sealing arrangement configured to provide a substantially airtight seal between the protective goggles and a face of a wearer, and
   a pump arranged to pump air into an ocular space defined by the protective goggles.
2. The protective goggles of clause 1, wherein the pump comprises a miniature pump, or a micropump, or a piezoelectric pump, or a piezoelectric miniature pump, or a piezoelectric micropump.
3. The protective goggles of clause 1 or 2, further comprising a visor or one or more eyepieces for protecting the wearer's eyes, wherein the ocular space is defined at least in part between the visor or one or more eyepieces and the sealing arrangement.
4. The protective goggles of any one of the preceding clauses, further comprising:
   a frame;
   wherein the visor or one or more eyepieces are connected to the frame; and
   wherein the ocular space is defined at least in part between the frame, the visor or one or more eyepieces, and the sealing arrangement.
5. The protective goggles of any one of the preceding clauses, wherein the pump is a piezoelectric micropump having an operating frequency of greater than 20 kHz.
6. The protective goggles of any one of the preceding clauses, further comprising a pressure sensor for sensing an air pressure in the ocular space, and a controller configured to control the pump responsive to the sensed air pressure.
7. The protective goggles of clause 6, further comprising a second pressure sensor for sensing an ambient air pressure, wherein the controller is configured to control the pump in response to a difference between the ocular space air pressure and the ambient air pressure.
8. The protective goggles of any one of clauses 1 to 5, further comprising a differential pressure sensor for measuring a difference between an ambient air pressure and an air pressure in the ocular space, and a controller configured to control the pump responsive to the measured difference in air pressure.
9. The protective goggles of any one of the preceding clauses, wherein the pump is fluidly connected to an air channel through which air is pumped into the ocular space, the air channel comprising an aperture configured to direct the air across an inside surface of a visor or one or more eyepieces of the protective goggles.
10. The protective goggles of clause 9, wherein the air channel is a channel within a frame of the protective goggles.
11. The protective goggles of any one of the preceding clauses, further comprising an outlet valve for allowing air out of the ocular space.
12. The protective goggles according to clause 11, wherein the outlet valve is configured to maintain the pressure in the ocular space at or above a predetermined pressure.
13. The protective goggles according to clause 11 or 12, wherein the outlet valve is configured to maintain a pressure differential between the ocular space and a surrounding atmosphere that is equal to or more than a predetermined pressure differential.
14. The protective goggles according to any one of the preceding clauses, further comprising a filter arranged to filter air pumped into the ocular space by the pump.
15. The protective goggles according to any one of the preceding clauses, further comprising: a strap or mounting arrangement for retaining the protective goggles in place.
16. The protective goggles according to any one of the preceding clauses, wherein the protective goggles are connectable to a nose cup of a mask, and wherein the pump is configured to pump air from within the nose cup of the mask into the ocular space of the protective goggles.

### 2. Active Defogging

1. A device comprising:
   a visor or eyepiece; and
   a defogging arrangement configured to generate and direct an airflow across an inside surface of the visor or eyepiece.
2. The device according to clause 1, wherein the device is configured to be mounted on a face or a head of a user.
3. The device according to clause 1 or 2, wherein the visor or eyepiece is optically clear.
4. The device according to any one of the preceding clauses, wherein the device is for protection, and/or vision enhancement, and/or displaying information.
5. The device of any one of the preceding clauses, wherein the defogging arrangement comprises an air channel through which the airflow passes, and an aperture in a wall of the air channel arranged to direct the airflow across the inside surface of the visor or eyepiece.
6. The device of clause 5, wherein the aperture comprises a nozzle for directing and/or accelerating the airflow.
7. The device according to clause 5 or 6, wherein the defogging arrangement comprises a plurality of the apertures in the wall of the air channel spaced apart along the air channel.
8. The device of any one of the preceding clauses, further comprising a frame, wherein the visor or eyepiece is connected to the frame and the air channel is a channel within the frame.
9. The device of any one of the preceding clauses, wherein the defogging arrangement comprises a fan, or a pump, or a piezoelectric pump, or a piezoelectric micropump, or a piezoelectric miniature pump, for generating the airflow.
10. The device of any one of the preceding clauses, further comprising a sealing arrangement, configured to provide a substantially airtight seal between the device and the face or head of a wearer.
11. The device of any one of the preceding clauses, further comprising a filter arranged to filter the airflow.
12. The device of any one of the preceding clauses, further comprising a strap or mounting arrangement for mounting the device on the face or head of a wearer.
13. The device of any one of the preceding clauses, wherein the device is protective goggles.

### 3. Goggles to Mask Connection

1. A respirator comprising:
   protective goggles for protecting a wearer's eyes; and
   a mask for covering a wearer's nose and mouth; wherein
      the protective goggles and the mask are detachably connectable to one another.
2. The respirator of clause 1, wherein the protective goggles and the mask are detachable from one another while in use, for example while being worn.
3. The respirator of clause 1 or 2, wherein the protective goggles comprise a first connector and the mask comprises a second connector, wherein the first connector and the second connector are detachably connectable together to detachably connect the protective goggles and the mask to one another.
4. The respirator according to clause 3, wherein a first one of the first connector and the second connector comprises a hook.
5. The respirator according to clause 4, wherein a second one of the first connector and the second connector comprises a catch or loop or ring or aperture or opening for connecting to the hook.
6. The respirator of clause 5, wherein the second one of the first connector and the second connector is a loop which extends outwardly away from the protective goggles or mask.
7. The respirator of any one of clauses 4 to 6, wherein the hook extends in a first direction away from the protective goggles or mask, and wherein an open portion of the hook faces in a second direction at least partly opposite to the first direction.
8. The respirator of any one of clauses 3 to 7, wherein the first connector and second connector are provided at a nose bridging portion of the protective goggles and the mask, respectively.
9. The respirator of any one of clauses 3 to 8, wherein the protective goggles and mask further comprise a magnetic connector configured to provide an additional detachable connection between the protective goggles and mask.

### 4. Close-Coupled Seal

1. A respirator comprising:
   protective goggles for protecting a wearer's eyes, comprising a goggle sealing arrangement configured to provide a seal between the protective goggles and the face of a wearer; and
   a mask for covering a wearer's nose and mouth, comprising a mask sealing arrangement configured to provide a seal between the mask and the face of a wearer,
   wherein the goggle sealing arrangement and the mask sealing arrangement comprise mutually overlapping portions.
2. The respirator of clause 1, wherein, in the mutually overlapping portions, the goggle sealing arrangement and the mask sealing arrangement have complementary, tessellating, or congruous shapes and/or tangential surfaces.
3. The respirator of clause 1 or 2, wherein, in the mutually overlapping portions, a sealing surface of the goggle sealing arrangement is substantially continuous with a sealing surface of the mask sealing arrangement, and/or tangential to a sealing surface of the mask sealing arrangement.
4. The respirator according to any one of the preceding clauses, wherein, in the mutually overlapping portions, a sealing surface of the goggle sealing arrangement and a sealing surface of the mask sealing arrangement are configured to form a continuous contact line or surface with the face of the wearer.
5. The respirator of any one of the preceding clauses, wherein the mutually overlapping portions are located at nasal bridge regions of the protective goggles and the mask.
6. The respirator of any one of the preceding clauses, wherein the mutually overlapping portions provide a layered structure.
7. The respirator of any one of the preceding clauses, wherein the mask sealing arrangement and/or the goggles sealing arrangement comprises one or more of:
   a resiliently deformable material; and
   an elastomeric material.
8. The respirator of any one of the preceding clauses, wherein, in the mutually overlapping portions, the mask sealing arrangement and the goggle sealing arrangement form a substantially airtight seal therebetween.

### 5. Nose Cup Chin Strap

1. A mask comprising:
   a nose cup for covering the wearer's nose and mouth; and
   a sealing arrangement configured to provide a seal between the mask and the face of the wearer,
   wherein the sealing arrangement comprises a chin portion which is configured to extend over a wearer's chin and has an outer surface which is configured to follow the shape of the wearer's chin.
2. The mask of clause 1, wherein:
   the nose cup is configured to extend from the top of the wearer's nose to the top of the wearer's chin, and
   the chin portion is configured to extend from the top of the wearer's chin to underneath the wearer's chin.
3. The mask of clause 1 or 2, wherein the chin portion is configured to conform to the shape of the wearer's chin.
4. The mask of any one of the preceding clauses, wherein a sealing surface of the chin portion is tangential to, or continuous with, a sealing surface of another part of the sealing arrangement.
5. The mask of any one of the preceding clauses, wherein the sealing arrangement comprises a protruding portion which protrudes from the chin portion to the nose cup.
6. The mask of clause 5, wherein the protruding portion partially overlaps the chin portion to form a recess.
7. The mask of clause 6, wherein the protruding portion is resiliently deformable and/or resiliently compressible.
8. The mask according to clause 6 or clause 7, wherein the recess is resiliently deformable and/or resiliently compressible.
9. The mask of any one of clauses 6 to 8, wherein the protruding portion comprises a bellows portion.
10. The mask of any one of clauses 6 to 9, wherein the recess is for receiving a chin strap of a helmet.
11. The mask of any one of the preceding clauses, wherein the nose cup is a quarter mask nose cup.
12. The mask according to any one of the preceding clauses, further comprising a tab connected to the chin portion for gripping or holding by a user.
13. The mask according to any one of the preceding clauses, further comprising a filter for filtering air breathed into the mask.

### 6. Single Action Filter Mount Connector

1. A connector for detachably mounting a filter on a mask, the connector comprising:
   a main body having an aperture through which air can flow;
   a resiliently deformable clip mechanism comprising jaws that are configured to engage a mounting portion, wherein the clip mechanism is resiliently deformable so that the jaws can be moved out of engagement with the mounting portion, and wherein the clip mechanism comprises an actuator that is movable relative to the main body to deform the clip mechanism to move the jaws.
2. The connector according to clause 1, wherein:
   the jaws are configured to engage the mounting portion therebetween,
   the clip mechanism is resiliently deformable so that the jaws can be moved or splayed apart; and
   the actuator is movable relative to the main body to deform the clip mechanism to move or splay the jaws apart.
3. The connector according to clause 1 or 2, wherein each of the jaws comprises a projecting lip or rim, for example an inwardly projecting rim or lip.
4. The connector according to any one of the preceding clauses, wherein each of the jaws has a chamfered lower surface.
5. The connector according to any one of the preceding clauses, wherein the clip mechanism comprises two jaws arranged to oppose each other.
6. The connector according to any one of the preceding clauses, wherein each of the jaws is curved.
7. The connector according to any one of the preceding clauses, wherein each of the jaws comprises a first end connected to the main body and a second end connected to the actuator.
8. The connector according to any one of the preceding clauses, wherein the jaws and the actuator are a unitary component and/or integral.
9. The connector according to any one of the preceding clauses, wherein the clip mechanism comprises a length of resiliently deformable material which is connected at each of its ends to the main body and which passes around the main body.
10. The connector according to clause 9 wherein the length of resiliently deformable material is connected at each of its ends to opposite ends of an arcuate section of the main body at an outer edge of the main body.
11. The connector according to any one of the preceding clauses, when the clip mechanism is squeezable so as to move the actuator relative to the main body.
12. The connector according to clause 11, wherein the clip mechanism is squeezable in a direction substantially perpendicular to a direction along which the jaws are configured to engage the mounting portion.
13. The connector according to any one of the preceding clauses, wherein the main body is a rigid body.
14. The connector according to any one of the preceding clauses, wherein the connector comprises a magnet.
15. A filter having the connector according to any one of the preceding clauses, wherein the jaws are configured to engage a mounting portion of a mask.
16. A mask having the connector according to any one of the preceding clauses, wherein the jaws are configured to engage a mounting portion of a filter.
17. The filter or mask according to clause 15 or 16, wherein the mounting portion comprises an upstanding member having a projecting lip or rim, for example an outwardly projecting lip or rim.
18. The filter or mask according to any one of clauses 15 to 17, wherein the connector and mounting portion both comprise magnets for magnetically connecting the connector to the mounting portion.

### 7. Negative Pressure Test Valve

1. A filter for a mask, the filter comprising:
   a filter connector for connecting the filter to a filter mount of the mask; and
   a valve having a first configuration in which the valve does not block flow of air through the filter connector, and a second configuration in which the valve blocks flow of air through the filter connector;
   wherein the valve is changeable from the first configuration to the second configuration by a user pressing the valve.
2. The filter according to clause 1, wherein the valve is built into, or integral with, or connected to, or part of, the filter connector.
3. The filter according to clause 1 or 2, wherein the valve is biased towards the first configuration when the valve is in the second configuration.
4. The filter according to any one of the preceding clauses, wherein the valve comprises:
   a valve seat that is connected to, or integral with, the filter connecter so that air can only flow through the filter connector via the valve seat; and
   a sealing member configured to contact the valve seat in the second configuration to seal the valve.
5. The filter according to clause 4 wherein the valve further comprises one or more biasing elements for biasing the sealing member apart from the valve seat.
6. The filter according to clause 4 or 5, wherein:
   in the first configuration the sealing member is spaced apart from the valve seat; and
   in the second configuration the sealing member is pressed into contact with the valve seat.
7. The filter according to clause 5 or 6, wherein the valve seat, the sealing member and the one or more biasing elements are integrally formed, and/or one-piece, and/or unitary, and/or a single part.
8. The filter according to any one of clauses 5 to 7, wherein the valve comprises a plurality of the biasing elements that are spaced apart around circumferences of the valve seat and sealing member so that air can flow between the one or more biasing elements when the valve is in the first configuration.
9. The filter according to any one of clauses 5 to 8, wherein the one or more biasing elements comprise resilient hinges, or living hinges, or integral hinges.
10. The filter according to any one of the preceding clauses, wherein the filter further comprises filter media arranged upstream of the filter connecter in an air flow through the filter.

### 8. Inflating Seal

1. A device comprising:
   a sealing arrangement configured to provide a seal between the device and the face of a wearer;
   wherein part of the sealing arrangement is inflatable to provide an improved seal between the device and the face of the wearer.
2. The device according to clause 1, wherein the sealing arrangement comprises an inflatable member that is inflatable to provide the improved seal between the device and the face of the wearer.
3. The device according to clause 2, wherein the inflatable member comprises an inflatable bladder, bag, or bellows.
4. The device according to any one of the preceding clauses, wherein part of the sealing arrangement that provides a seal between the device and the nose of the wearer is inflatable
5. The device according to any one of the preceding clauses, comprising a pump, for example a piezoelectric micropump, arranged to pump air into the part of the sealing arrangement.
6. The device according to clause 5, comprising a controller configured to control an operation of the pump.
7. The device according to clause 6, wherein the controller is configured to control the pump to inflate the part of the sealing arrangement while the device is being worn by a wearer.
8. The device according to clause 6 or 7, wherein the controller is configured to control the pump to pump air into the part of the sealing arrangement in response to an operation of the wearer.
9. The device according to any one of clauses 6 to 8, wherein:
   the device comprise a sensor for sensing a pressure in the part of the sealing arrangement, or a pressure in a sealed space of the device such as an ocular space, or a pressure difference between a sealed space of the device such as an ocular space and the surroundings of the device; and
   wherein the controller is configured to control an operation of the pump based on an output of the pressure sensor.
10. The device according to any one of the preceding clauses, wherein the part of the sealing arrangement comprises a valve which is operable to deflate the part of the sealing arrangement.
11. The device according to clause 10, wherein the controller is configured to control the valve.
12. The device according to any one of the preceding clauses, wherein the device is a head or face mountable device.
13. The device according to any one of the preceding clauses, wherein the device is protective goggles or a mask.
14. The device according to any one of the preceding clauses, wherein the device is protective goggles, and wherein the protective goggles further comprise a frame and a visor or one or more eyepieces connected to the frame for protecting a wearer's eyes.
15. A device comprising:
   a sealing arrangement configured to provide a seal between the device and the face of a wearer;
   a strap or mounting arrangement for attaching the device to the head or face of the wearer; and
   an inflatable element that is arranged to apply outward pressure to the strap or mounting arrangement when inflated to provide an improved seal between the device and the face of the wearer.
16. The device according to clause 15, wherein the device is a head or face mountable device.
17. The device according to clause 15 or 16, wherein the device is protective goggles or a mask.

### 9. Disposable Filter Used as Mask Harness

1. A flexible mounting arrangement or harness for a mask, comprising:
   a plenum comprising a flexible air permeable structure; and
   a connector for connecting the mounting arrangement or harness to a filter mount of the mask, wherein the connector is in fluid communication with the plenum;
   wherein the plenum is enclosed, or substantially enclosed, at least partly by filter material.
2. The flexible mounting arrangement or harness according to clause 1, wherein the plenum is enclosed, or substantially enclosed, by filter material.
3. The flexible mounting arrangement or harness according to any one of the preceding clauses, wherein the flexible mounting arrangement or harness comprises a laminate structure comprising the air permeable structure sandwiched between an inner layer and an outer layer, and wherein the inner layer and/or the outer layer comprises filter material.
4. The flexible mounting arrangement or harness according to any one of the preceding clauses, wherein the air permeable structure is resilient.
5. The flexible mounting arrangement or harness according to any one of the preceding clauses, wherein the air permeable structure comprises one or more of:
   a 3D spacer material;
   a 3D spacer fabric;
   expanded paper;
   an open cell foam;
   a 3D printed lattice; and
   a chainmail.
6. The flexible mounting arrangement or harness according to any one of the preceding clauses, wherein the filter material comprises one or more of:
   a fluoroplastic porous film;
   an electret;
   a spunbound or meltblown material; and
   expanded PTFE.
7. The flexible mounting arrangement or harness according to any one of the preceding clauses, wherein the mounting arrangement or harness is configured to extend from the mask along both sides of a wearer's head to the back of the wearer's head.
8. The flexible mounting arrangement or harness according to any one of the preceding clauses, further comprising one or more fasteners for fastening the flexible mounting arrangement or harness around the wearer's head.
9. The flexible mounting arrangement or harness according to any one of the preceding clauses, further comprising a layer of material on an external surface of the filter material.
10. The flexible mounting arrangement or harness according to clause 9, wherein the layer of material is hydrophobic and/or has a hydrophobic layer, coating or finish.
11. A mask comprising:
   a filter mount; and
   the mounting arrangement or harness of any one of the preceding clauses connected to the filter mount.

### 10. Mask Harness as External Air Source Connection

1. A flexible mounting arrangement or harness for a mask, comprising:
   a plenum comprising a flexible air permeable structure;
   a first connector for connecting the mounting arrangement or harness to a filter mount of the mask, wherein the first connector is in fluid communication with the plenum;
   a second connector for connecting the mounting arrangement or harness to an air supply, wherein the second connector is in fluid communication with the plenum; and
   a gas impermeable material enclosing, or substantially enclosing, the plenum.
2. The flexible mounting arrangement or harness according to clause 1, wherein the gas impermeable material is a gas and liquid impermeable material.
3. The flexible mounting arrangement or harness according to clause 1 or 2, wherein the gas impermeable material comprises one or more of:
   an elastomeric material;
   rubber, for example thermoset or thermoplastic rubber;
   a sealed fabric;
   polyurethane; and
   Butyl rubber.
4. The flexible mounting arrangement or harness according to any one of the preceding clauses, wherein the second connector is for connecting the flexible mounting arrangement or harness to a breathing hose.
5. The flexible mounting arrangement or harness according to any one of the preceding clauses, wherein the flexible mounting arrangement or harness comprises a laminate structure comprising the air permeable structure sandwiched between an inner layer of gas impermeable material and an outer layer of gas impermeable material.
6. The flexible mounting arrangement or harness according to any one of the preceding clauses, wherein the air permeable structure is resilient.
7. The flexible mounting arrangement or harness according to any one of the preceding clauses, wherein the air permeable structure comprises one or more of:
   a 3D spacer material;
   a 3D spacer fabric;
   expanded paper;
   an open cell foam;
   a 3D printed lattice; and
   a chainmail.
8. The flexible mounting arrangement or harness according to any one of the preceding clauses, wherein the mounting arrangement or harness is configured to extend from the mask along both sides of a wearer's head to the back of the wearer's head.
9. The flexible mounting arrangement or harness according to any one of the preceding clauses, further comprising one or more fasteners for fastening the flexible mounting arrangement or harness around the wearer's head.
10. A mask comprising:
   a filter mount; and
   the mounting arrangement or harness of any one of the preceding clauses connected to the filter mount.

1. A mask comprising:
a nose cup for covering a wearer's nose and mouth; and
a sealing arrangement configured to provide a seal between the mask and the wearer's face;
wherein the sealing arrangement comprises a chin portion configured to extend over the wearer's chin and an outer surface configured to follow the shape of the wearer's chin.

2. The mask of claim 1, wherein:
the nose cup is configured to extend from the top of the wearer's nose to the top of the wearer's chin;
the chin portion is configured to extend from the top of the wearer's chin to underneath the wearer's chin; and
the chin portion is optionally configured to conform to the shape of the wearer's chin.

3. (canceled)

4. The mask of claim 1, wherein a sealing surface of the chin portion is or continuous with a sealing surface of another part of the sealing arrangement.

5. The mask of claim 1, wherein the sealing arrangement comprises a protruding portion which protrudes from the chin portion to the nose cup.

6. The mask of claim 5, wherein the protruding portion partially overlaps the chin portion to form a recess.

7. The mask of claim 6, wherein the protruding portion is at least one of resiliently deformable and resiliently compressible, and optionally wherein the recess is at least one of resiliently deformable and resiliently compressible.

8. (canceled)

9. The mask of claim 6, wherein the protruding portion comprises a bellows portion.

10. The mask of claim 6, wherein the recess is for receiving a chin strap of a helmet.

11. The mask of claim 1, wherein the nose cup is a quarter mask nose cup.

12. The mask of claim 1, further comprising a tab connected to the chin portion for gripping or holding by a user.

13. The mask of claim 1, further comprising a filter for filtering air breathed into the mask.

14. A respirator comprising:
a mask comprising a nose cup for covering a wearer's nose and mouth, and a sealing arrangement configured to provide a seal between the mask and the wearer's face, wherein the sealing arrangement comprises a chin portion configured to extend over the wearer's chin and an outer surface configured to follow the shape of the wearer's chin; and
protective goggles for protecting the wearer's eyes, wherein the protective goggles are detachably connectable to the mask.

15. The respirator of claim 14, wherein the protective goggles comprise a first connector and the mask comprises a second connector, wherein the first connector and the second connector are detachably connectable together to detachably connect the protective goggles to the mask.

16. The respirator of claim 15, wherein:
one of the first connector and the second connector comprises a hook;
the other of the first connector and the second connector comprises a catch for connecting to the hook;
the hook optionally extends outwardly from the protective goggles or mask in a first direction with an open portion of the hook facing in a second direction at least partially opposite to the first direction; and
the catch optionally extends outwardly from the protective goggles or mask.

17. (canceled)

18. (canceled)

19. The respirator of claim 15, wherein the first connector and the second connector are provided at a nose bridging portion of the protective goggles and the mask, respectively.

20. The respirator of claim 15, wherein the protective goggles and mask further comprise a magnetic connector configured to provide an additional detachable connection between the protective goggles and mask.

21. The respirator of claim 14, wherein:
the protective goggles comprise a goggle sealing arrangement configured to provide a seal between the protective goggles and the wearer's face;
the mask comprises a mask sealing arrangement configured to provide a seal between the mask and the wearer's face; and
the goggle sealing arrangement and the mask sealing arrangement comprise mutually overlapping portions.

22. The respirator of claim 21, wherein, in the mutually overlapping portions, a sealing surface of the goggle sealing arrangement is continuous with a sealing surface of the mask sealing arrangement.

23. The respirator of claim 21, wherein, in the mutually overlapping portions, a sealing surface of the goggle sealing arrangement and a sealing surface of the mask sealing arrangement are configured to form a continuous contact line with the wearer's face.

24. The respirator of claim 21, wherein the mutually overlapping portions are located at nasal bridge regions of the protective goggles and the mask.
